(19) United States
(12) Patent Application Publication
(10) Pub. No.: US 2026/0301869 A1
(43) Pub. Date: Oct. 1, 2026

(54) Systems and Methods for Predicting DNA Structural Stability Using Lagrangian Density

(71) Applicant: Jonathan Washburn, Austin, TX (US)

(72) Inventor: Jonathan Washburn, Austin, TX (US)

(21) Appl. No.: 19/631,820

(22) Filed: Mar. 27, 2026

Related U.S. Application Data
(60) Provisional application No. 63/780,226, filed on Mar. 29, 2025.

(51) Int. Cl. G16B 30/00

(52) U.S. Cl. CPC G16B 30/00

(57) ABSTRACT

A first-principles framework for predicting structural stability of deoxyribonucleic acid (DNA) constructs is disclosed. A sequence stability metric, sequence extent, helical pitch, groove ratio, and Recognition Science-derived parameters are used to derive coefficients of a DNA Lagrangian density acting on a local base-pairing probability field over a helical coordinate domain. The DNA Lagrangian density includes a kinetic contribution associated with spatial rigidity and a potential contribution associated with local pairing preference and helical periodic modulation. An Euler-Lagrange equation associated with the DNA Lagrangian density is solved to generate a structural stability profile for the DNA construct. One or more stability metrics, including a structural stability metric and a melting-barrier metric, may be computed from the profile, and a structural stability result may be output for validation, ranking, filtering, mutation assessment, archival candidate selection, therapeutic construct evaluation, or integration into a DNA design workflow.

### CROSS-REFERENCE TO RELATED APPLICATIONS

This application claims benefit of priority to U.S. Provisional Patent Application No. 63/780,226, titled “A Recognition Physics-Derived Lagrangian Density Formulation for Predicting DNA Structural Stability” filed on Mar. 29, 2025.

### BACKGROUND

The present disclosure relates generally to computational molecular biology, biophysics, and DNA engineering, and more particularly to systems and methods for predicting structural stability of deoxyribonucleic acid (DNA) constructs using a field-based, first-principles formulation. More specifically, the disclosure concerns a Recognition Science-derived Lagrangian-density framework for evaluating sequence-dependent and geometry-dependent stability of DNA molecules, including stability as a function of sequence-related pairing strength, helical pitch, groove-related geometry, and derived stability metrics.

Conventional approaches for evaluating DNA stability often rely on empirical nearest-neighbor thermodynamic tables, fitted melting models, or large-scale force-field simulations. While such approaches can be useful in some contexts, they may depend heavily on experimental calibration, may not provide a compact first-principles account of core stability parameters, and may be difficult to transfer cleanly across different candidate constructs, geometric regimes, or design workflows. Accordingly, there remains a need for a technically grounded and computationally tractable framework that models DNA structural stability using a compact governing formulation tied to physically interpretable sequence and geometric inputs, and that can generate actionable outputs for validation, ranking, and downstream DNA design applications.

### SUMMARY

The present disclosure provides a first-principles framework for modeling and predicting structural stability of deoxyribonucleic acid (DNA) constructs using a Recognition Science-derived Lagrangian density. In disclosed embodiments, a DNA construct is represented as a coupled sequence-and-geometry object characterized by a sequence stability metric, a sequence extent, a helical pitch, a groove ratio, and one or more derived parameters. A DNA Lagrangian density is constructed to act on a local base-pairing probability field over a helical coordinate domain, the Lagrangian density including a kinetic contribution associated with spatial rigidity and a potential contribution associated with local pairing preference and helical periodic modulation. An Euler-Lagrange equation associated with the DNA Lagrangian density is then solved or otherwise evaluated to generate a structural stability profile for the DNA construct, from which one or more stability metrics and a structural stability result may be derived.

In one embodiment, a computer-implemented method receives or determines sequence-related and geometry-related inputs for a candidate DNA construct, derives model parameters including a stiffness parameter, a sequence-scaled pairing coefficient, and a periodic modulation coefficient, constructs the DNA Lagrangian density, and solves a boundary-value problem over a finite helical interval subject to paired-end boundary conditions. The resulting local base-pairing probability field may be used to determine a periodic amplitude, a structural stability metric, and a melting-barrier metric, and the candidate DNA construct may then be accepted, rejected, ranked, or otherwise evaluated according to one or more structural-stability criteria.

In another embodiment, the disclosed formulation is implemented in a DNA structural stability prediction system including one or more processors, memory, parameter derivation logic, solver logic, metric computation logic, and output logic. Such a system may operate as a standalone computational tool or as a stability-evaluation engine within a broader design workflow, including comparative sequence analysis, mutation assessment, archival DNA candidate selection, therapeutic construct validation, or integration into a DNARP pipeline. In this manner, the present disclosure provides a technically grounded and computationally tractable approach for converting sequence-derived and geometry-derived DNA inputs into actionable structural-stability outputs.

In disclosed embodiments, the method, system, and computer-readable medium aspects share a common inventive concept: a stability-evaluation engine that (i) derives or receives sequence-related and geometry-related inputs, (ii) constructs a DNA Lagrangian density acting on a local base-pairing probability field over a finite helical domain, (iii) solves or evaluates an associated Euler-Lagrange boundary-value formulation to obtain a stability profile C(r), and (iv) computes one or more stability metrics used to output a structural stability result that automatically controls selection, validation, ranking, or rejection of candidate DNA constructs.

In some embodiments, the structural stability result is used as a technical control output in a DNA screening or design workflow by automatically filtering, rejecting, ranking, or selecting candidate constructs prior to downstream processing. In contrast to approaches that rely primarily on empirically fitted nearest-neighbor tables or computationally intensive force-field simulations, the disclosed framework derives a compact coefficient set from physically interpretable sequence and geometry inputs, solves a finite-domain boundary-value problem to generate a field profile C(r), and computes one or more stability metrics (including a periodic amplitude A and one or more derived metrics) that drive an automated accept/reject/rank outcome. For example, a candidate may be classified as acceptable only when one or more acceptance criteria are satisfied, including an amplitude bound, one or more metric thresholds, and/or geometric envelope constraints, thereby converting the solved stability profile and derived metrics into a machine-actionable decision suitable for computational validation and design integration.

### BRIEF DESCRIPTION OF THE DRAWINGS

The various advantages of the examples will become apparent to one skilled in the art by reading the following specification and appended claims, and by referencing the following drawings, in which:

FIG. 1 is a schematic block diagram of an example DNA structural stability prediction system configured to receive sequence-related and geometry-related inputs, derive model parameters, construct a DNA Lagrangian density, solve for a structural stability profile, and generate a structural stability output.

FIG. 2 is a schematic representation of an example DNA helical coordinate model showing a DNA duplex defined over a helical coordinate domain having a sequence length, a helical pitch, a groove-related geometric descriptor, and a local base-pairing probability field.

FIG. 3 is a schematic diagram of an example Lagrangian-density component architecture showing a DNA Lagrangian density including a kinetic contribution, a potential contribution, and associated parameter contributions including stiffness, sequence-scaled pairing, periodic modulation, and optional groove-sensitive correction.

FIG. 4 is a flow diagram of an example DNA stability prediction workflow including receiving sequence-related and geometry-related inputs, deriving model parameters, constructing a DNA Lagrangian density, solving an Euler-Lagrange equation, generating a stability profile, computing one or more stability metrics, and producing a structural stability result.

FIG. 5 is a graph of an example representative stability profile showing a baseline paired condition, a bounded periodic deviation of a local base-pairing probability field, and finite-domain end-effect behavior.

FIG. 6 is a schematic acceptance-region diagram showing an example design space partitioned into accepted, rejected, and transition regions according to one or more stability-related criteria.

FIG. 7 is a schematic block diagram of an example computing system configured to implement the disclosed DNA structural stability formulation, including parameter derivation, solver, metric computation, decision, storage, communication, and output functionality.

FIG. 8 is a flow diagram of an example mutation comparison workflow in which a reference sequence and a variant sequence are evaluated to generate comparative stability profiles, a differential stability measure, and a mutation impact result.

FIG. 9 is a schematic diagram of an example DNARP integration system in which a DNA stability evaluation engine operates within a candidate-generation and optimization pipeline to filter, rank, or validate candidate constructs.

FIG. 10 is a schematic diagram of an example application-specific output system configured to provide one or more structural-stability outputs including design validation, mutation assessment, archival candidate selection, therapeutic construct validation, candidate ranking, and report generation.

### DETAILED DESCRIPTION

The present invention provides a first-principles formulation for modeling and predicting structural stability of deoxyribonucleic acid (DNA) molecules using a Recognition Science-derived Lagrangian density. In particular, the invention defines a DNA structural stability prediction system (100) configured to receive sequence-related and geometry-related inputs through an input interface (110), derive model parameters from those inputs in a parameter derivation module (160), construct a DNA-specific Lagrangian in a Lagrangian construction module (170), solve for a resulting stability behavior in a solver module (180), and provide one or more stability outputs through an output module (190), as shown in FIG. 1.

In disclosed embodiments, the input interface (110) receives at least a sequence stability input (120), a helical pitch input (130), a groove ratio input (140), and one or more RS-derived parameter inputs (150). The sequence stability input (120) may encode sequence-dependent pairing strength, compositional stability, or other sequence-derived quantities relevant to duplex persistence. The helical pitch input (130) may encode a pitch value, a pitch constraint, or a pitch-derived design parameter associated with a modeled DNA helix. The groove ratio input (140) may encode a major-to-minor groove relationship or another groove-related descriptor associated with the geometry of the duplex. The RS-derived parameter inputs (150) may include one or more first-principles quantities used to define coefficients of the disclosed Lagrangian-density formulation.

Using these inputs, the parameter derivation module (160) generates one or more model parameters used in the disclosed stability formulation, including parameters associated with stacking rigidity, sequence-scaled pairing energy, periodic helical modulation, and, in some embodiments, groove-sensitive correction behavior. The Lagrangian construction module (170) then forms a DNA Lagrangian density from those parameters as a function of a local base-pairing field and one or more geometric variables. The solver module (180) determines a resulting stability profile by solving, or approximately solving, the governing equation associated with the constructed Lagrangian. In this manner, the disclosed system (100) predicts structural stability from the internal form of the formulation itself rather than relying exclusively on empirical melting tables or purely fitted thermodynamic lookups.

In some embodiments, the output module (190) provides a profile describing local base-pairing behavior along the DNA molecule, one or more stability metrics derived from that profile, and a resulting determination regarding structural acceptability, predicted persistence, relative robustness, or ranking among candidate constructs. The output may therefore include, for example, a predicted field profile, an amplitude or deviation measure, a structural stability metric, a melting-related barrier metric, or a stable/unstable decision. In further embodiments, the output module (190) may provide a rank-ordered result across multiple candidate DNA constructs or may furnish an acceptance or rejection signal for downstream design selection.

The invention is therefore directed not merely to an abstract mathematical description, but to a concrete computational framework for DNA structural stability assessment in which physically meaningful inputs are transformed into a modeled stability behavior and then into an actionable technical output. Because the disclosed framework is built around a DNA-specific Lagrangian-density formulation, it can be used as a stability-analysis engine for design validation, mutation assessment, archival sequence evaluation, therapeutic construct screening, or other DNA-focused computational workflows. In some embodiments, the system (100) may operate as a component within a broader design environment in which candidate sequences and candidate geometric constraints are evaluated prior to synthesis, storage, or therapeutic use.

At a high level, the invention predicts DNA structural stability by minimizing, or otherwise evaluating, an action constructed from the disclosed Lagrangian density, thereby producing a modeled local base-pairing behavior over the DNA domain and one or more stability-related outputs. This architecture provides a direct bridge between first-principles parameter derivation and practical DNA stability prediction, while preserving a modular implementation in which the input interface (110), parameter derivation module (160), Lagrangian construction module (170), solver module (180), and output module (190) may be implemented together or in any suitable distributed combination within the DNA structural stability prediction system (100).

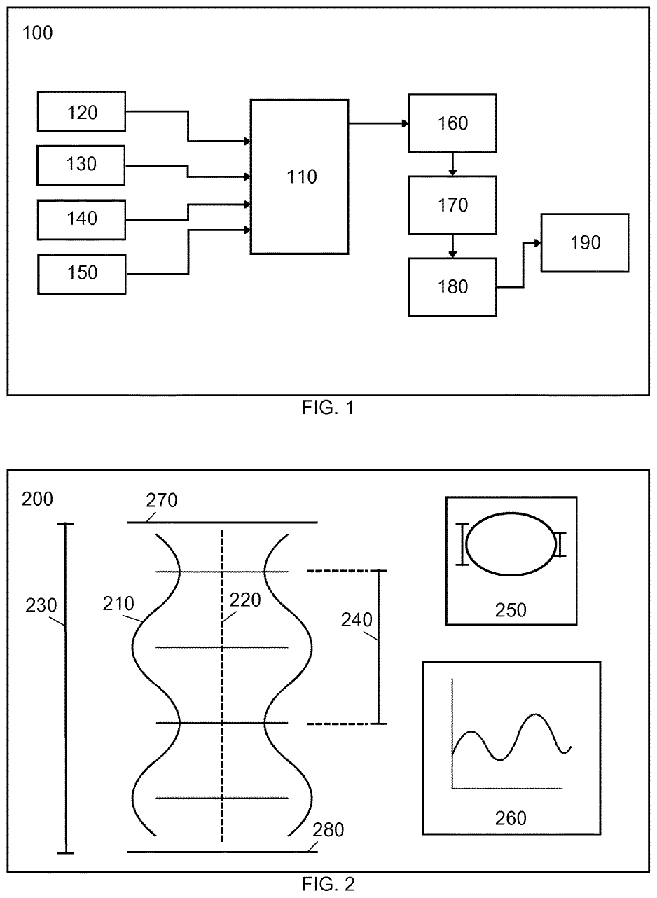

For purposes of the present disclosure, a local base-pairing probability field is denoted as C(r), where r is a helical coordinate defined along a DNA duplex (210) of a DNA helical coordinate model (200), as illustrated in FIG. 2. In disclosed embodiments, C(r) represents a local measure of duplex closure or pairing persistence at position r, with larger values corresponding to greater local structural integrity and smaller values corresponding to increased local destabilization or opening tendency. In preferred embodiments, C(r) is bounded on the interval [0,1], such that C(r)=1 corresponds to a fully paired local condition and C(r)=0 corresponds to a fully unpaired local condition. Intermediate values of C(r) may represent partial pairing, reduced persistence, or a locally weakened pairing state.

The coordinate r is a continuous or effectively continuous helical coordinate defined along a helical coordinate axis (220). The coordinate axis (220) extends between a first end boundary (270) and a second end boundary (280) of the modeled DNA domain. A sequence length is denoted by L and corresponds to the total modeled extent of the DNA duplex (210) along the helical coordinate axis (220). In currently disclosed embodiments, the sequence length (230) may be related to the number of base pairs n_bp by the relation L=n_bp*3.4 Angstrom, such that each base pair contributes an average axial extent of approximately 3.4 Angstrom to the modeled duplex length. The value n_bp therefore denotes the number of base pairs represented within the modeled sequence.

A helical pitch is denoted by P and corresponds to the axial distance associated with one helical turn of the DNA duplex (210), as indicated by the helical pitch (240). A reference pitch scale is denoted by P0 and may be used in disclosed embodiments as an RS-derived pitch scale, a reference helical-turn scale, or a comparison pitch for parameter derivation and stability evaluation. The pitch P may be supplied as an input, imposed as a design constraint, varied during optimization, or evaluated relative to P0 in order to assess whether a candidate DNA geometry remains within an acceptable structural regime.

A groove ratio is denoted by G and corresponds to a geometric descriptor of groove asymmetry or major-to-minor groove relationship within the DNA duplex (210), as indicated by groove ratio (250). A reference groove ratio is denoted by G0 and may represent a preferred, nominal, or RS-derived groove ratio used for comparison, normalization, or groove-correction analysis. In some embodiments, G0 is taken as phi. The groove ratio G may be treated as part of the geometric state of the duplex and may be used to determine whether a candidate structure departs from a preferred groove configuration.

A sequence stability metric is denoted by S_stab and represents a sequence-dependent quantity associated with duplex persistence, pairing strength, or resistance to destabilization. In some embodiments, S_stab may be derived from nucleotide composition, base-pair content, or another sequence-dependent stability measure. For example, S_stab may reflect the relative contribution of CG-rich and AT-rich sequence content, such that larger values of S_stab correspond to a deeper effective pairing well and a greater predicted resistance to strand separation. The sequence stability metric may therefore serve as a compact input variable that captures sequence-dependent stability information without requiring that every sequence contribution be independently reintroduced into the governing formulation.

Additional variables used in the present disclosure include X_DNA, E_coh, E_bp, a_bp, kappa_DNA, lambda_DNA, beta, and gamma. The parameter X_DNA denotes a characteristic DNA length scale used in disclosed embodiments for deriving one or more model coefficients. The quantity E_coh denotes a coherence-related energy scale used in deriving stiffness and modulation quantities. The quantity E_bp denotes a base-pairing energy scale used in disclosed embodiments to determine a sequence-scaled pairing contribution. The quantity a_bp denotes an effective base-pair spacing or base-pair length scale and, in certain embodiments, is defined as a_bp=X_DNA/4. The parameter kappa_DNA denotes a stiffness parameter associated with the gradient cost or stacking-rigidity contribution of the formulation. The parameter lambda_DNA denotes a sequence-scaled potential coefficient associated with the effective pairing contribution. The parameter beta denotes a periodic modulation coefficient associated with helical forcing or pitch-dependent modulation. The parameter gamma denotes a groove-correction coefficient that may be used in optional embodiments to account for departures of G from G0.

A periodic amplitude is denoted by A and represents the magnitude of a helical modulation in the resulting stability profile. In disclosed embodiments, A may quantify the size of a periodic deviation of C(r) from a nominally stable baseline. Smaller values of A therefore correspond to smaller oscillatory deviations and generally indicate greater structural stability within the linearized model. One or more derived stability metrics may also be used. A structural stability metric is denoted by M_struct and may quantify deviation of the local base-pairing probability field from an idealized stable condition. A melting-barrier metric is denoted by M_melt and may quantify an energy-related barrier associated with full strand separation. A damage-resistance metric is denoted by M_damage and may, in some embodiments, characterize resistance to perturbation or degradation as a function of the modeled stability state.

Where DNARP notation is used, a program or construct may be denoted by D=(S, H, E), where S denotes a sequence component, H denotes a shape component, and E denotes an effect, execution, expression, or application-related component depending on implementation context. In such embodiments, the disclosed formulation may operate on, validate, constrain, or rank one or more aspects of S and H, including sequence-derived stability and shape-derived parameters such as pitch and groove ratio.

Unless otherwise indicated, the local base-pairing probability field (260) is evaluated over the interval r in [0,L] subject to boundary conditions at the first end boundary (270) and the second end boundary (280). In currently disclosed embodiments, the boundary conditions are taken as C(0)=C(L)=1, thereby representing a modeled sequence whose ends are fixed or constrained to a paired state for purposes of solving the disclosed stability formulation. These definitions provide a consistent notation for the subsequent description of the DNA Lagrangian density, its parameter derivation, the resulting Euler-Lagrange equation, and the stability metrics and acceptance criteria derived therefrom.

Recognition Science is used in the present disclosure in a limited and targeted manner to support the disclosed DNA structural stability formulation and the specific constants and relations expressly applied within that formulation. As used herein, “Recognition Science-derived” parameters refer to quantities derived using the recognition cost functional J(x) and the disclosed scale relation involving phi, including E_coh and any coefficients computed from E_coh that are used to form or parameterize the DNA Lagrangian density (including kappa_DNA and beta in disclosed embodiments). The present invention is not directed to Recognition Science as a general platform or to unrelated theoretical domains; instead, Recognition Science is incorporated only to the extent needed to provide a first-principles basis for the specific DNA Lagrangian-density framework described herein.

In disclosed embodiments, Recognition Science provides a constrained cost structure from which the constants used in the DNA formulation are derived. A foundational relation is the Recognition Composition Law, which may be written in ASCII-safe form as J(x*y)+J(x/y)=2*J(x)*J(y)+2*J(x)+2*J(y), where J(x) is a recognition cost functional defined over positive scale ratios. Within the Recognition Science framework used here, this relation, together with normalization and calibration conditions, yields a unique cost functional of the form J(x)=0.5*(x+1/x)−1. This cost functional supplies the first-principles basis for treating recognition departure, scale deviation, and stability departure in a way that is internally constrained rather than empirically fitted.

In further disclosed embodiments, the Recognition Science framework forces a preferred scale relation involving phi, where phi denotes the golden ratio. The quantity phi is used in the present disclosure only as a theory-derived scale constant supporting the disclosed energy, geometric, and periodicity relations. In this limited context, phi provides a basis for deriving a coherence-related energy scale E_coh, which in disclosed embodiments is taken as E_coh=phi\hat{ }−5. This coherence-related energy scale is then used downstream in the derivation of one or more parameters of the DNA Lagrangian density, including the stiffness parameter kappa_DNA and the periodic modulation coefficient beta.

Recognition Science further provides an 8-tick or 2\hat{ }D periodic support structure in the case D=3, which is used in the present disclosure only as limited support language for the disclosed periodic and geometric treatment of DNA. In particular, the Recognition Science framework identifies a minimal period of 2\hat{ }D, which yields an 8-fold structural support at D=3. This 8-fold support is not introduced here as an independent invention, but instead as a limited theoretical basis for why discrete periodic structure and forced scale regularity may be expected in a DNA-related formulation built from Recognition Science-derived quantities.

Where useful for explaining the periodic coupling term, the present disclosure may also refer to an action-related or scale-linking relation in which hbar is expressed through E_coh and a corresponding Recognition Science time scale. In such embodiments, hbar=E_coh*tau0 may be used as part of the explanatory chain for the disclosed estimate of beta, thereby linking coherence energy, structural pitch scale, and bounded periodic forcing. This relation is used only to support the disclosed periodic modulation coefficient and is not intended to broaden the invention beyond the specific DNA stability formulation recited herein.

A DNA-focused bridge is also provided in order to tie the limited Recognition Science foundation to the actual structure of the disclosed invention. In disclosed embodiments, the double-helix framing may be described as accommodating eight recognition slots within an 8-position structural interpretation, and codon-related structure may be described within an 8×8 recognition space. These statements are included only as supportive context for the DNA-specific use of the disclosed constants and periodicity assumptions. Likewise, the Recognition Science manuscript support for a first-principles DNA chain culminating in approximately 10.5 bp/turn is incorporated here only as corroborative support for the helical periodicity discussion used in connection with pitch P and reference pitch P0. That manuscript support is not introduced as a separate claimed invention, but rather as confirmatory support that the disclosed pitch-related treatment of DNA is consistent with a first-principles Recognition Science derivation.

Accordingly, the Recognition Science material included in the present disclosure is deliberately limited to the origin of J(x), the forced scale relation involving phi, the derivation and role of E_coh, the bounded periodicity support associated with the 8-tick framework, and the DNA-specific bridge needed to support the disclosed helical pitch and related constants. These limited Recognition Science elements provide the first-principles support necessary for the present DNA structural stability formulation while maintaining the focus of the specification on the disclosed Lagrangian density, the resulting stability profile, and the practical computational prediction of DNA structural stability.

As shown in FIG. 2, the DNA duplex (210) is modeled as a helical domain extending along a helical coordinate axis (220) between a first end boundary (270) and a second end boundary (280). The modeled extent of the duplex along that axis is the sequence length (230), denoted by L, and the local state of pairing along the duplex is represented by the local base-pairing probability field (260), denoted by C(r). In this framework, the coordinate r provides a continuous positional variable over the interval r in [0,L], thereby allowing the structural state of the duplex to be treated as a spatially varying field over a finite helical domain rather than as a purely discrete list of independent base-pair events. This geometric setup is used so that spatial variation, helical periodicity, end constraints, and sequence-dependent stability can all be incorporated into a single continuous formulation.

The sequence length (230) defines the axial extent of the modeled DNA domain and therefore fixes the interval over which the disclosed Lagrangian density is evaluated. In disclosed embodiments, the sequence length L is related to the number of base pairs n_bp according to L=n_bp*3.4 Angstrom, such that the duplex is treated as having an average axial step of approximately 3.4 Angstrom per base pair. This relation provides a practical bridge between a physical DNA sequence and the continuous helical domain used in the formulation. Accordingly, when a candidate sequence is supplied for evaluation, the sequence length may be determined directly from the number of base pairs in that sequence, and the resulting value of L defines the spatial interval over which the field C(r) is solved or otherwise evaluated.

The helical geometry of the duplex is further characterized by the helical pitch (240), denoted by P, and the groove ratio (250), denoted by G. The helical pitch P represents the axial distance associated with one helical turn of the DNA duplex (210), while the groove ratio G represents a geometric descriptor of groove asymmetry or relative major-to-minor groove configuration within the duplex. These quantities together define a shape input for the modeled DNA structure. In particular, P determines the periodic spacing of the helical modulation used in the disclosed potential term, while G provides a geometric quantity that may be used, in optional embodiments, to account for departures from a preferred groove configuration. Thus, the shape state of the duplex is not treated as an abstract or external annotation, but as a set of explicit geometric inputs that directly enter the stability formulation.

Because the disclosed model treats the duplex as a helical domain rather than a simple linear chain, pitch and groove geometry are used as structural descriptors that influence stability behavior through the form of the Lagrangian density. A candidate DNA construct may therefore be evaluated not only by its sequence-dependent pairing tendency, but also by whether its helical pitch and groove configuration remain within a structurally admissible regime. In some embodiments, P may be supplied as an imposed design constraint or a candidate geometry to be tested, while G may be supplied as an observed or selected groove-related descriptor. In further embodiments, one or both of P and G may be varied during a design or optimization process in order to identify a more stable structural configuration for a given sequence.

The sequence stability metric S_stab captures sequence-dependent pairing strength in a compact form suitable for use in the disclosed formulation. Rather than requiring every local sequence feature to be reintroduced independently into the governing equations, S_stab acts as a sequence-derived quantity representing the overall pairing persistence, duplex robustness, or resistance to destabilization associated with a candidate sequence. In some embodiments, S_stab may be based on nucleotide composition, including relative CG-rich and AT-rich character, while in other embodiments it may be based on a broader sequence-derived stability measure used by a design environment or validation workflow. Larger values of S_stab correspond to greater effective pairing strength and therefore to a deeper sequence-scaled pairing contribution in the disclosed potential. In this manner, the formulation preserves a direct link between sequence content and structural stability while avoiding unnecessary expansion of the field model into a large set of sequence-specific empirical coefficients.

In one illustrative embodiment, the sequence stability metric S_stab is computed directly from nucleotide composition of the candidate sequence. For example, S_stab may be computed as a weighted composition score based on counts or fractions of CG and AT content over a selected sequence interval, such as S_stab=w_CG·f_CG+w_AT·f_AT, where f_CG and f_AT represent respective CG and AT fractions (or respective counts normalized by sequence length), and w_CG and w_AT are selected weighting constants for a given application profile. In another illustrative embodiment, S_stab is provided by an upstream design workflow or validation environment as a normalized stability score mapped into a bounded numeric range used by the disclosed formulation. The present disclosure is not limited to any one S_stab calculation, provided that S_stab provides a sequence-derived measure that increases with increased effective pairing strength and thereby increases the sequence-scaled pairing contribution described herein.

The geometric and sequence-related quantities used in the present disclosure are therefore linked in a coordinated input model. The sequence length L defines the modeled domain, the helical pitch P defines the periodic spacing of the helical structure, the groove ratio G defines a geometric descriptor of groove configuration, and the sequence stability metric S_stab defines a sequence-derived measure of pairing strength. Together, these quantities provide the principal inputs used to construct the disclosed DNA stability model. As a result, the invention can evaluate a DNA construct as a coupled sequence-and-shape object, in which both sequence-derived stability and helical geometry contribute to the resulting structural assessment.

In some embodiments, a reference pitch scale P0 is used as an RS-derived pitch quantity for comparison, calibration, or parameter derivation. The presently disclosed material includes an embodiment in which P0=35.6 Angstrom, and also includes an embodiment in which 4*phi\hat{ }2 is used as an RS helical-turn reference. These descriptions are harmonized in the present disclosure as corroborative support for the helical periodicity discussion rather than as separate inventions. More specifically, P0 may be treated as a reference helical-turn scale used in the disclosed parameter chain, while a value such as 4*phi\hat{ }2 may be understood as an RS-related scale expression supporting the same general periodicity framework. The updated manuscript support indicating a first-principles DNA chain culminating in approximately 10.5 bp/turn is likewise incorporated here only as corroborative support for the pitch-related treatment of DNA and for the proposition that the disclosed helical periodicity assumptions are consistent with a first-principles derivation. In this way, the present disclosure maintains a single DNA stability invention while using multiple mutually supportive descriptions of helical periodicity to reinforce the physical basis for pitch-dependent modeling.

Accordingly, the DNA input model and geometric setup disclosed herein treat a DNA molecule as a finite helical domain having explicit length, pitch, groove, and sequence-stability inputs. This setup provides the geometric and sequence-dependent foundation for the DNA Lagrangian density described below and ensures that the resulting formulation is anchored to concrete physical descriptors of the duplex rather than to an abstract field lacking structural interpretation. The definitions associated with the DNA helical coordinate model (200), including the DNA duplex (210), helical coordinate axis (220), sequence length (230), helical pitch (240), groove ratio (250), local base-pairing probability field (260), first end boundary (270), and second end boundary (280), therefore supply the geometric framework on which the subsequent stability formulation operates.

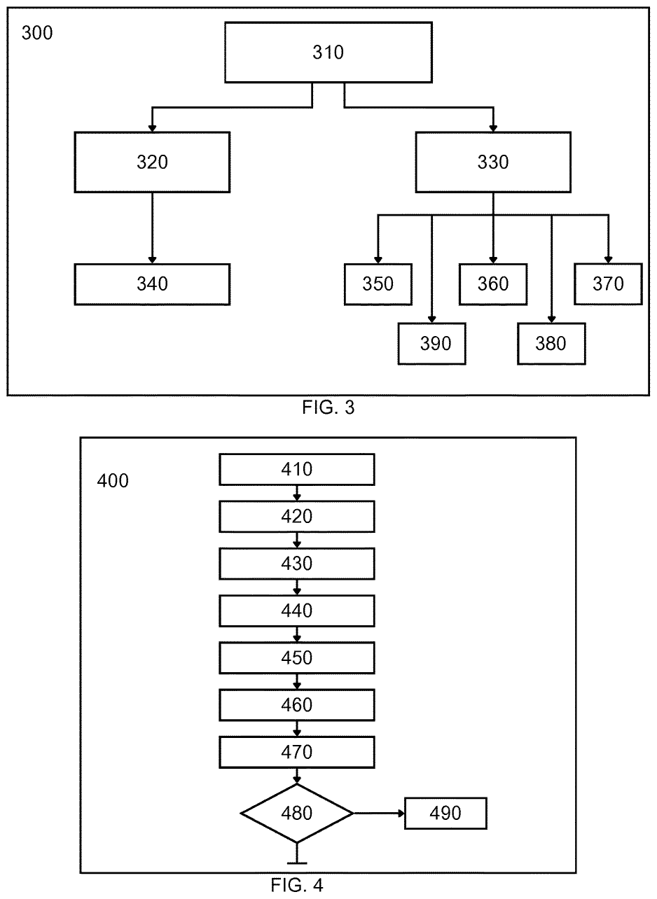

The disclosed DNA structural stability formulation is built around a DNA Lagrangian density (310) having the form L_DNA=T(C′)+V_Lag(C, r; P, G, S_stab), as illustrated by the Lagrangian density component architecture (300) of FIG. 3. In this formulation, the DNA Lagrangian density (310) is defined as the sum of a kinetic term (320) and a potential term (330). The formulation acts on the local base-pairing probability field C(r) over the helical domain previously described, such that structural stability is determined from the spatial behavior of the field under the disclosed energetic and geometric constraints. By expressing DNA stability in this form, the invention provides a single governing density from which the stable profile, associated metrics, and resulting structural decisions can be derived.

In disclosed embodiments, the full equation set is:

$$L\_DNA = T ( {C}^{′} ) + V\_Lag ( C , r ; P , G , S\_stab )$$

$$T ( {C}^{′} ) = ( kappa\_DNA / 2 ) * ( dC / dr ) \hat{} 2$$

$$V\_Lag ( C , r ; P , G , S\_stab ) = lambda\_DNA ( S\_stab ) * ( ( 1 - C ) \hat{} 2 / 2 - beta * ( 1 - C ) * cos ( 2 * pi * r / P ) ) + gamma * ( 1 - G / G 0 ) \hat{} 2 * C \hat{} 2$$

As shown in FIG. 3, the DNA Lagrangian density (310) includes the kinetic term T(C′) (320) and the potential term V_Lag (330), with parameter contributions including stiffness parameter kappa_DNA (340), sequence-scaled potential coefficient lambda_DNA (350), periodic modulation coefficient beta (360), groove correction coefficient gamma (370), groove reference value G0 (380), and helical phase term (390). The disclosed formulation is therefore not an unconstrained symbolic expression, but a structured stability model in which each term has a defined physical role and a defined relation to the modeled DNA duplex.

The kinetic term (320), given by T(C′)=(kappa_DNA/2)*(dC/dr)\hat{ }2, penalizes spatial variation of the local base-pairing probability field. Physically, this term represents the energetic cost of allowing the pairing state to vary too sharply along the helical coordinate. A stable duplex does not ordinarily undergo arbitrarily abrupt transitions between fully paired and destabilized states at neighboring positions; instead, such variations are moderated by stacking rigidity, backbone coupling, and the continuity of the duplex as a helical structure. The stiffness parameter kappa_DNA (340) therefore controls the degree to which rapid spatial fluctuations in C(r) are suppressed. Larger values of kappa_DNA correspond to stronger resistance against abrupt local deformation of the pairing field, while smaller values allow greater spatial variation. The kinetic term (320) is a mandatory component of the disclosed core formulation because it provides the gradient-cost contribution that distinguishes a spatially structured duplex from a collection of independent local pairing states.

The potential term (330) represents the local energetic preference of the duplex for a paired configuration together with a helical periodic modulation and, in optional embodiments, a groove-sensitive correction. The base pairing portion of the potential is given by lambda_DNA(S_stab)*((1−C)\hat{ }2/2). This component defines a local pairing well centered at C=1, thereby favoring a state in which the DNA duplex remains locally paired. When C approaches 1, this contribution approaches zero, reflecting a preferred stable condition. As C departs from 1, the contribution increases, reflecting the energetic cost of reduced pairing persistence or strand opening tendency. The sequence-scaled potential coefficient lambda_DNA (350) therefore determines the effective depth and strength of this pairing preference. Because lambda_DNA depends on S_stab, the formulation directly incorporates sequence-derived stability into the local pairing well without requiring a large empirical lookup structure.

The periodic modulation portion of the potential term is given by −lambda_DNA(S_stab)*beta*(1−C)*cos(2*pi*r/P). This term introduces helical periodicity into the stability model by modulating the local pairing tendency as a function of position along the duplex. The periodic modulation coefficient beta (360) governs the strength of this helical forcing, while the helical phase term (390), expressed as cos(2*pi*r/P), imposes periodicity according to the helical pitch P. Physically, this periodic contribution reflects that the DNA duplex is not a featureless linear rod, but a helical structure whose geometry introduces a repeating positional relation along the molecular axis. The periodic term therefore allows the formulation to encode the fact that structural stability is shaped not only by local pairing preference, but also by the repeating geometry of the helix. In disclosed embodiments, beta remains bounded and functions as a modulation factor rather than as a dominant destabilizing term, such that the primary stable tendency of the system continues to be governed by the pairing well centered at C=1.

The optional groove-related correction is given by gamma*(1−G/G0)\hat{ }2*C\hat{ }2. This term is included to account for the possibility that departures of the groove ratio G from a reference groove ratio G0 may introduce a secondary geometric influence on structural stability. The groove correction coefficient gamma (370) scales the magnitude of this contribution, while the groove reference value G0 (380) defines the preferred or nominal groove relation against which G is compared. Because the factor (1−G/G0)\hat{ }2 vanishes when G=G0, the groove-sensitive contribution disappears at the reference groove condition. Accordingly, the groove term is not mandatory to the core formulation and is treated in the present disclosure as an optional embodiment refinement. Its purpose is to preserve support for embodiments in which groove asymmetry contributes measurably to the local energy landscape, while allowing the principal invention to remain centered on the kinetic term, the sequence-scaled pairing term, and the helical periodic modulation.

The disclosed DNA Lagrangian density therefore contains two mandatory structural components and one optional refinement. The mandatory components are the kinetic term (320) and the base potential structure contained within the potential term (330), including the sequence-scaled pairing well and the pitch-dependent periodic modulation. These mandatory components are sufficient to define the core invention because they provide, respectively, a spatial continuity penalty, a local stability preference centered on the paired state, and a helical modulation linked to DNA geometry. The optional refinement is the groove-sensitive correction associated with gamma (370) and G0 (380). This optional term may be used where added geometric sensitivity is desired, but omission of that term does not remove the central functionality of the formulation.

From a physical perspective, the disclosed equation set may be understood as balancing three effects. First, the kinetic term opposes abrupt spatial changes in the pairing field, thereby favoring smooth structural behavior along the duplex. Second, the sequence-scaled pairing well favors local persistence of a paired state, with the strength of that preference depending on the sequence stability metric S_stab. Third, the helical periodic contribution modulates that pairing preference according to the geometric periodicity imposed by pitch P. When included, the groove correction provides a fourth, secondary effect that penalizes departures from a preferred groove ratio. The stable DNA profile predicted by the invention is therefore the result of a constrained balance among spatial smoothness, sequence-dependent pairing strength, helical periodicity, and, in some embodiments, groove-related geometry.

This structure also explains why the disclosed formulation is particularly suitable for first-principles DNA stability prediction. Rather than relying on experimentally fitted nearest-neighbor tables or a large collection of empirical force-field parameters, the DNA Lagrangian density (310) expresses stability through a compact set of interpretable terms whose coefficients are derived from theory-grounded quantities and sequence-or geometry-related inputs. The stiffness parameter kappa_DNA (340) governs spatial resistance to fluctuation, the sequence-scaled potential coefficient lambda_DNA (350) governs the effective pairing strength, the periodic modulation coefficient beta (360) governs the extent of helical forcing, and the optional groove correction coefficient gamma (370) governs the magnitude of groove-sensitive refinement. As a result, the formulation preserves direct physical interpretability while remaining computationally tractable.

The DNA Lagrangian density (310) is therefore the core operative expression of the present invention. It provides the formal mechanism by which a candidate DNA construct, characterized by sequence-derived stability and helical geometry, is converted into a solvable stability model. Once constructed, this density may be used to define an action over the helical domain, and minimization or evaluation of that action yields the governing equation for the local base-pairing probability field. The disclosed formulation thus serves as the central bridge between the DNA input model previously described and the stability profile, metrics, and design decisions described below. In this sense, FIG. 3 provides the conceptual architecture of the formulation, while the equation set above provides the operative mathematical statement of the DNA structural stability model.

With continued reference to FIG. 3, the stiffness parameter kappa_DNA (340) is the coefficient that sets the gradient-cost contribution of the kinetic term T(C′) (320). In physical terms, kappa_DNA quantifies the resistance of the DNA pairing field to abrupt spatial variation along the helical coordinate. A locally paired base pair at one position influences neighboring positions through stacking interactions, backbone continuity, and the coupled character of the duplex, such that sharp changes in the local base-pairing probability field are energetically disfavored. The role of kappa_DNA is therefore to encode stacking rigidity in the disclosed field formulation and to determine how strongly the system penalizes rapid spatial changes in C(r).

In disclosed embodiments, kappa_DNA is derived from a coherence-related energy scale and a characteristic DNA length scale according to the relation kappa_DNA=E_coh*X_DNA. This relation reflects that the gradient cost arises from the expenditure of a recognition-related or coherence-related energy across a characteristic DNA structural length. The quantity E_coh provides the energy scale associated with coherent recognition behavior, while X_DNA provides the corresponding DNA length scale over which that energetic influence is applied. Their product therefore yields a stiffness parameter having the dimensions appropriate for weighting the spatial derivative term (dC/dr)\hat{ }2 in the Lagrangian density. This relation is consistent with the physical interpretation that fluctuations in the local pairing state must traverse a finite DNA length under a finite energetic cost, and that the resulting rigidity of the field should scale with both of those quantities.

In certain disclosed embodiments, the more general relation may be written as kappa_DNA=c1*E_coh*X_DNA, where c1 is a dimensionless geometric factor. In the presently disclosed one-dimensional recognition-chain embodiment, c1=1, such that no additional geometric correction is applied and the stiffness parameter reduces to kappa_DNA=E_coh*X_DNA. Stated differently, the disclosed 1D chain treatment takes the minimal-overhead geometric factor to be unity, so the stiffness coefficient is directly proportional to the product of the coherence energy scale and the characteristic DNA length scale. This embodiment is useful because it keeps the core formulation compact and theory-grounded while preserving room for alternative dimensional or geometric refinements in other implementations.

The units of kappa_DNA follow directly from this derivation. Because E_coh is an energy quantity and X_DNA is a length quantity, kappa_DNA has units of energy times length. This unit structure is appropriate for the coefficient of (dC/dr)\hat{ }2, since the derivative dC/dr carries inverse-length dependence and the kinetic term T(C′)=(kappa_DNA/2)*(dC/dr)\hat{ }2 therefore contributes an energy-density quantity over the modeled helical domain. In disclosed numerical embodiments, E_coh may be taken as approximately 0.09 eV and X_DNA may be taken as approximately 13.6 Angstrom, yielding kappa_DNA=1.224 eV*Angstrom. The same quantity may be expressed in SI units as approximately 1.96×10\hat{ }−29 J*m. These numerical values are illustrative of one disclosed derivation path and are not required in every embodiment, but they demonstrate that the derived stiffness parameter has a concrete physical scale rather than being an unconstrained fitting constant.

The stiffness parameter kappa_DNA is positive in the disclosed embodiments. This positivity is important both physically and mathematically. Physically, a positive kappa_DNA means that spatial fluctuations in the local base-pairing probability field carry a non-negative energetic cost, such that the system resists uncontrolled oscillation or arbitrarily sharp structural defects. Mathematically, positivity of kappa_DNA ensures that the kinetic term (320) is non-negative and that the formulation remains stable with respect to gradient perturbations. If kappa_DNA were zero or negative, the field would lose the disclosed rigidity behavior and the kinetic contribution would fail to serve its intended role as a stabilizing gradient-cost term. The disclosed relation kappa_DNA=E_coh*X_DNA, together with positive E_coh and positive X_DNA, therefore provides a direct basis for the positivity of kappa_DNA.

The derivation of kappa_DNA also reinforces why the present formulation differs from empirically fitted DNA-stability models. In nearest-neighbor thermodynamic schemes, statistical models, or force-field simulations, a rigidity-related quantity is typically introduced through experimentally calibrated parameters or through a large parameter set inferred from data. By contrast, the present disclosure derives kappa_DNA from the first-principles chain E_coh and X_DNA, such that the gradient-cost contribution is not merely assigned as a fitting coefficient but instead follows from the disclosed Recognition Science-derived parameter structure. In this way, kappa_DNA serves as a theory-grounded stiffness term linking coherence-related energy and DNA structural length to the spatial smoothness of the local pairing field.

Accordingly, kappa_DNA (340) is the parameter that converts first-principles energetic and structural scales into a concrete gradient penalty within the DNA Lagrangian density (310). Through the kinetic term (320), it enforces spatial smoothness, encodes stacking rigidity, and helps ensure that the predicted stability profile varies in a physically meaningful manner along the DNA duplex. The disclosed derivation kappa_DNA=E_coh*X_DNA, optionally expressed as kappa_DNA=c1*E_coh*X_DNA with c1=1 in the presently disclosed 1D chain embodiment, therefore provides a compact and operationally useful basis for the stiffness parameter used throughout the remainder of the specification.

With continued reference to FIG. 3, the sequence-scaled potential coefficient lambda_DNA (350) is the parameter that sets the strength of the effective pairing well within the potential term V_Lag (330). In physical terms, lambda_DNA determines how strongly the disclosed formulation favors the locally paired condition C=1 over a destabilized or unpaired condition. Whereas kappa_DNA governs the energetic cost of spatial variation in the field, lambda_DNA governs the local energetic preference for maintaining duplex pairing. The role of lambda_DNA is therefore to convert sequence-dependent stability information into a pairing-energy contribution that can be directly used in the DNA Lagrangian density (310).

In disclosed embodiments, lambda_DNA is derived by first defining an effective base-pair axial scale a_bp according to the relation a_bp=X_DNA/4. The quantity a_bp represents an effective axial step associated with the characteristic DNA length scale X_DNA, and serves as the normalization length used to convert a base-pairing energy into a pairing-energy density or pairing-energy-per-unit-length contribution. Using this definition, the sequence-scaled potential coefficient is given by lambda_DNA(S_stab)=(E_bp/a_bp)*S_stab. Thus, lambda_DNA is derived from a base-pairing energy scale E_bp, normalized by the effective axial spacing a_bp, and then scaled by the sequence stability metric S_stab.

This derivation has a clear physical interpretation. The ratio E_bp/a_bp may be understood as an effective pairing-energy-per-unit-length quantity. Because E_bp is a base-pairing energy scale and a_bp is an axial spacing, their ratio expresses the energetic strength associated with pairing over a unit length of the DNA domain. Multiplication by S_stab then adapts that baseline pairing-energy density to the actual sequence under consideration. In this way, lambda_DNA is not merely a free coefficient inserted into the potential well, but rather a theory-grounded and sequence-responsive parameter that expresses how strongly a particular sequence tends to remain paired. The potential well term lambda_DNA(S_stab)*((1−C)\hat{ }2/2) therefore becomes deeper or shallower depending on the sequence-derived stability represented by S_stab.

The dependence of lambda_DNA on S_stab is monotonic in the disclosed embodiments. Because E_bp and a_bp are treated as positive quantities, increasing S_stab increases lambda_DNA, while decreasing S_stab decreases lambda_DNA. This monotonicity is important because it preserves the intended physical meaning of the formulation: sequences that are more stable should produce a stronger effective pairing contribution, and sequences that are less stable should produce a weaker one. Accordingly, the disclosed relation lambda_DNA(S_stab)=(E_bp/a_bp)*S_stab ensures that the sequence-scaled potential coefficient increases with increasing sequence stability metric. This property is also consistent with the theorem support described for lambda_dna_pos, lambda_scales_stab, and lambda_proportional.

The positivity of lambda_DNA follows naturally from the same derivation. For S_stab>0, and with positive E_bp and positive a_bp, the coefficient lambda_DNA is positive. This means that the term lambda_DNA(S_stab)*((1−C)\hat{ }2/2) defines a proper local pairing well having a minimum at C=1 and increasing energetic cost as C departs from the fully paired state. A positive lambda_DNA is therefore essential to the disclosed formulation because it ensures that the potential term actually favors pairing rather than destabilization. If lambda_DNA were zero, the sequence-dependent pairing preference would vanish. If it were negative, the local potential would invert and would no longer model structural stability in the manner disclosed herein.

The sequence stability metric S_stab may encode sequence-dependent stability in a variety of technically appropriate ways while preserving the disclosed relation for lambda_DNA. In some embodiments, S_stab reflects nucleotide composition or relative base-pair content, including distinctions between CG-rich and AT-rich sequence character. Because CG-rich regions generally correspond to stronger effective pairing persistence than AT-rich regions, a CG-rich sequence may be assigned a larger S_stab value, thereby producing a larger lambda_DNA and a deeper effective pairing well. Conversely, an AT-rich sequence may be assigned a smaller S_stab value, thereby producing a smaller lambda_DNA and a shallower pairing well. These examples are illustrative of the disclosed monotonic mapping between sequence-derived stability and the sequence-scaled potential coefficient, and do not limit the invention to any single procedure for computing S_stab.

The derivation of lambda_DNA also supports the broader first-principles character of the invention. Conventional DNA stability models often rely on large empirical parameter tables, nearest-neighbor fits, or experimentally tuned force-field values to determine how strongly a sequence resists opening. By contrast, the present disclosure defines lambda_DNA from a compact derivation chain: a characteristic DNA length scale X_DNA yields a_bp=X_DNA/4, a base-pairing energy scale E_bp supplies the energetic numerator, and the sequence stability metric S_stab supplies the sequence-specific scaling. As a result, the pairing-well strength is expressed in a way that is interpretable, sequence-responsive, and not dependent on experimentally fitted melting tables.

Accordingly, lambda_DNA (350) is the parameter that converts sequence-derived stability into a local pairing-energy density within the DNA Lagrangian density (310). Through the relation a_bp=X_DNA/4 and the disclosed derivation lambda_DNA(S_stab)=(E_bp/a_bp)*S_stab, the invention obtains a positive and monotone sequence-scaled potential coefficient that deepens the pairing well for more stable sequences and shallows the pairing well for less stable sequences. In this manner, lambda_DNA provides the operative bridge between sequence content and the local energetic preference for duplex persistence in the disclosed structural stability formulation.

With continued reference to FIG. 3, the periodic modulation coefficient beta (360) is the parameter that sets the strength of the helical forcing contribution within the potential term V_Lag (330). In the disclosed formulation, beta scales the term-lambda_DNA(S_stab)*(1−C)*cos(2*pi*r/P), and therefore governs the extent to which the local pairing tendency is modulated by helical periodicity along the DNA duplex. Physically, beta does not define the existence of the pairing well itself, but instead determines how strongly the helical structure of the duplex perturbs or modulates that pairing preference as a function of position. The role of beta is therefore to encode pitch-linked periodic structure in a bounded manner so that the resulting field remains primarily pairing-stabilized while still reflecting the repeating geometry of the helix.

In disclosed embodiments, beta is derived from a Recognition Science-based scale relation linking the coherence-related energy scale E_coh to a helical reference length. One disclosed relation is beta=E_coh*P0/(hbar*c), where P0 is a reference pitch scale, hbar is an action-related constant expressed in the Recognition Science parameter chain, and c is a propagation constant used to maintain dimensional consistency in the coupling relation. This expression provides a first-principles basis for the periodic modulation coefficient by tying the strength of helical forcing to the product of a coherence-energy scale and a DNA-relevant pitch scale, normalized by a corresponding action-and-propagation denominator. In this way, beta is not introduced as an arbitrary oscillation parameter, but as a bounded coupling coefficient arising from the same Recognition Science-derived constant structure that supports the remainder of the disclosed formulation.

The physical interpretation of this derivation is that beta measures the relative strength of helical periodic forcing compared to the underlying coherence-governed scale of the modeled system. The numerator E_coh*P0 captures the product of a coherence-related energetic scale and a characteristic helical length scale, while the denominator hbar*c normalizes that quantity against a corresponding action-propagation scale. As a result, beta expresses a dimensionless measure of how strongly the repeating helical structure should modulate the otherwise pairing-centered local potential. Because the DNA duplex is helical rather than purely linear, the local pairing environment is not identical at all positions along the coordinate axis. The coefficient beta captures that periodic structural effect while keeping it subordinate to the primary pairing-well behavior governed by lambda_DNA.

In disclosed embodiments, beta is bounded such that 0<beta<1. This boundedness is important both physically and mathematically. Physically, it ensures that the periodic modulation remains a secondary forcing term rather than a dominant destabilizing contribution that would overwhelm the local pairing preference centered at C=1. Mathematically, bounded beta helps preserve the stability of the linearized solution and maintains the amplitude of the resulting periodic response within a controlled range. A value of beta approaching zero corresponds to very weak helical modulation, while larger but still subunit values correspond to stronger helical influence that nonetheless remains subordinate to the principal pairing-well structure. The disclosed boundedness of beta is therefore part of the design of the formulation itself and is not merely a numerical convenience.

One disclosed base estimate yields beta_base approx 0.016, demonstrating that the direct Recognition Science-derived periodic forcing may be relatively small in magnitude. In some embodiments, an effective periodic modulation coefficient beta_eff may be used to account for collective, structural, or environmental amplification of the basic periodic coupling while still preserving the bounded nature of the parameter. A disclosed illustrative value is beta_eff approx 0.052. Such an effective coefficient remains within the bounded interval 0<beta<1 and therefore continues to function as a modulation factor rather than as a dominant instability driver. The present disclosure accordingly preserves support both for a direct base derivation of beta and for an effective bounded coupling embodiment that better captures aggregate helical modulation in practical implementations.

The role of beta in the disclosed potential term can be understood by considering the factor cos(2*pi*r/P), identified in FIG. 3 as the helical phase term (390). The phase term imposes a positional periodicity along the helical coordinate, with period set by the helical pitch P. The coefficient beta determines how strongly that periodicity influences the local energetic preference associated with pairing. Accordingly, beta does not independently create the periodic structure, since that periodicity is set by P through the cosine phase term. Instead, beta weights the extent to which the periodic geometry affects the local stability landscape. In this sense, the helical phase term (390) supplies the positional pattern, while the periodic modulation coefficient beta (360) supplies the magnitude of the corresponding forcing.

The bounded nature of beta is also reflected in the resulting stability profile. Because the periodic response amplitude A is proportional to beta in the disclosed linearized solution, maintaining beta within a small positive interval ensures that the predicted field C(r) remains close to the stable baseline for structurally acceptable sequences and geometries. This is consistent with the intended physical interpretation of the invention, namely that DNA structural stability is fundamentally governed by a strong local pairing tendency modulated by a comparatively smaller helical periodic effect. If beta were not bounded, the periodic term could dominate the profile in a manner inconsistent with the disclosed stable-duplex regime. The derivation and treatment of beta therefore help preserve both the physical realism and the computational tractability of the model.

The derivation of beta further distinguishes the present invention from purely empirical or phenomenological oscillation models. In conventional approaches, a periodic coefficient might be inserted to improve fit or to mimic an observed helical effect without a theory-grounded derivation chain. By contrast, the present disclosure ties beta to the Recognition Science-derived coherence scale E_coh, the reference pitch scale P0, and the action-propagation relation involving hbar and c. As a result, the periodic modulation coefficient is not simply selected to match data, but instead follows from a disclosed first-principles parameter structure that is consistent with the overall DNA Lagrangian-density framework.

Accordingly, beta (360) is the bounded periodic modulation coefficient that converts helical periodicity into a controlled positional forcing term within the DNA Lagrangian density (310). Through the disclosed relation beta=E_coh*P0/(hbar*c), and through optional use of an effective bounded coefficient where appropriate, the invention obtains a dimensionless parameter that scales the influence of the helical phase term (390) without overwhelming the local pairing preference defined by lambda_DNA. In this manner, beta provides the operative bridge between the first-principles Recognition Science parameter chain and the pitch-dependent modulation of DNA structural stability used throughout the remainder of the specification.

With continued reference to FIG. 3, the groove correction coefficient gamma (370) provides an optional geometric refinement to the potential term V_Lag (330) through the contribution gamma*(1−G/G0)\hat{ }2*C\hat{ }2. This contribution is not required to practice the core invention, but may be included in embodiments in which groove geometry is treated as a secondary structural factor affecting local DNA stability. The principal purpose of the groove-correction term is to preserve support for embodiments in which departures of the groove ratio G from a reference groove ratio G0 contribute an additional energetic penalty, while keeping the central invention focused on the mandatory pairing-well and helical-modulation components of the DNA Lagrangian density (310).

In disclosed embodiments, the groove ratio G corresponds to a geometric descriptor of groove asymmetry or major-to-minor groove relation of the DNA duplex, and the reference groove ratio G0 (380) defines a preferred or nominal groove condition against which the actual groove ratio is compared. In certain embodiments, G0=phi, such that the reference groove condition is set by the Recognition Science-derived scale constant phi. Under this definition, the factor (1−G/G0)\hat{ }2 measures a squared relative departure of the actual groove ratio from the reference groove ratio. Because the departure is squared, the correction is non-negative and depends on magnitude of deviation rather than sign, so groove ratios above and below the reference value may both produce a positive correction when they differ from G0.

The structure of the groove-related term is significant. The coefficient gamma (370) scales the magnitude of the correction, while the factor (1−G/G0)\hat{ }2 determines how strongly the correction is activated by a deviation of G from G0. Multiplication by C\hat{ }2 makes the contribution dependent on the local pairing state, such that the groove-related penalty remains tied to the local structural state of the duplex rather than acting as a detached constant offset. In this way, the groove term may be understood as a geometry-sensitive refinement to the local energy landscape, one that becomes relevant when groove configuration departs from a preferred relation but remains subordinate to the principal pairing and helical terms of the formulation.

A notable property of the disclosed groove correction is that it vanishes when G=G0. When the groove ratio equals the reference groove ratio, the factor (1−G/G0)\hat{ }2 becomes zero, and the entire contribution gamma*(1−G/G0)\hat{ }2*C\hat{ }2 disappears. This vanishing behavior is important because it confirms that the groove term is truly corrective rather than foundational. The core DNA structural stability formulation therefore remains fully defined without any added groove penalty at the reference groove condition. Stated differently, the disclosed invention does not require nonzero groove correction in order to operate; instead, the correction appears only when the actual groove geometry departs from the reference groove relation.

In some embodiments, gamma is selected as a comparatively small coefficient so that the groove-related contribution remains a secondary refinement relative to the pairing-well term and the periodic modulation term. One disclosed illustrative embodiment takes gamma=E_coh/X_DNA, which provides a compact first-principles scaling for the groove correction. Under representative disclosed values E_coh approx 0.09 eV and X_DNA approx 13.6 Angstrom, this yields gamma approx 0.0066 eV/Angstrom. This magnitude is materially smaller than the principal sequence-scaled pairing coefficient in many operative embodiments and therefore supports the intended role of the groove term as a limited correction rather than a dominant driver of structural stability.

A disclosed small-magnitude example further illustrates the optional and subordinate character of this groove-sensitive contribution. For a representative case in which G=1.70 and G0=phi=1.618, the deviation factor is (1−G/G0)\hat{ }2=(1−1.70/1.618)\hat{ }2, which is approximately (0.051)\hat{ }2=0.0026. Using the illustrative value gamma approx 0.0066 eV/Angstrom, the resulting correction is approximately 0.0066*0.0026=1.7×10\hat{ }−5 eV/Angstrom before multiplication by C\hat{ }2. Because C(r) is bounded in [0,1], multiplication by C\hat{ }2 does not increase this magnitude. This example shows that, within the disclosed groove-ratio window, the groove-correction term may be very small relative to the principal stability contributions.

This small-magnitude behavior is consistent with the disclosed operational range in which groove ratio variation within G in [1.5, 1.7] has negligible effect on the overall DNA Lagrangian density compared with the dominant influence of sequence stability S_stab and helical pitch P. In such embodiments, the main structural stability drivers remain the stiffness-controlled gradient term, the sequence-scaled pairing well, and the bounded helical modulation. The groove term is therefore useful as a refinement for preserving geometric sensitivity and fallback claim support, but it does not alter the central conclusion that the principal stability behavior is governed by sequence-derived pairing strength and pitch-linked periodicity.

The optional character of this embodiment is important for prosecution readiness and claim flexibility. By expressly identifying the groove correction as optional, the present disclosure preserves support for independent claims that omit groove sensitivity entirely, as well as dependent claims that recite a groove-sensitive correction term, a reference groove ratio G0, or a particular choice such as G0=phi. This drafting posture allows the specification to remain tightly focused on the central invention while still providing explicit written-description support for narrower geometric refinements if needed during prosecution.

Accordingly, the contribution gamma*(1−G/G0)\hat{ }2*C\hat{ }2 is disclosed as an optional groove-sensitive refinement to the potential term V_Lag (330). In embodiments where it is included, the groove correction coefficient gamma (370) and reference groove ratio G0 (380) introduce a non-negative geometric penalty that vanishes at G=G0 and remains small within the disclosed groove operating window. The groove-sensitive term therefore preserves support for additional geometric fidelity without changing the fact that the core invention is centered on the mandatory kinetic term, the sequence-scaled pairing well, and the bounded pitch-dependent periodic modulation of the DNA Lagrangian density (310).

Starting from the disclosed DNA Lagrangian density (310), the governing equation for the local base-pairing probability field C(r) is obtained by applying the Euler-Lagrange condition to the action defined over the helical DNA domain. The local base-pairing probability field is treated as a spatial field over the coordinate interval r in [0,L], and the stable profile of that field is determined by requiring that the corresponding action be stationary with respect to admissible variations in C(r). In the present disclosure, this variational step provides the operative bridge between the structural form of the DNA Lagrangian density and the actual stability profile predicted for a candidate DNA construct.

For a Lagrangian density of the form L_DNA=L_DNA(C, C′, r), where C′=dC/dr, the governing condition is

$$d / dr ( partial L\_DNA / partial {C}^{′} ) - partial L\_DNA / partial C = 0$$

This Euler-Lagrange relation states that the stable field configuration is the one for which the first variation of the action vanishes. In the present invention, that condition is applied to the component relationships shown in FIG. 3 over the helical DNA domain defined in FIG. 2. More specifically, the kinetic term (320), the potential term (330), the stiffness parameter kappa_DNA (340), the sequence-scaled potential coefficient lambda_DNA (350), the periodic modulation coefficient beta (360), and the helical phase term (390) together determine the differential equation governing the field C(r) along the helical coordinate axis (220).

Using the disclosed core equation set,

$$L\_DNA = T ( {C}^{′} ) + V\_Lag ( C , r ; P , G , S\_stab )$$

$$T ( {C}^{′} ) = ( kappa\_DNA / 2 ) * ( dC / dr ) \hat{} 2$$

$$V\_Lag ( C , r ; P , G , S\_stab ) = lambda\_DNA ( S\_stab ) * ( ( 1 - C ) \hat{} 2 / 2 - beta * ( 1 - C ) * cos ( 2 * pi * r / P ) ) + gamma * ( 1 - G / G 0 ) \hat{} 2 * C \hat{} 2$$

the derivative of the kinetic term with respect to C′ is

$$partial L\_DNA / partial {C}^{′} = kappa\_DNA * ( dC / dr )$$

and therefore

$$d / dr ( partial L\_DNA / partial {C}^{′} = kappa\_DNA * ( d \hat{} 2 C / dr \hat{} 2 )$$

The derivative of the potential term with respect to C contains the local pairing-well contribution, the periodic modulation contribution, and, where included, the optional groove-sensitive correction. In the core linearized treatment emphasized in the present disclosure, the resulting governing equation may be written as

$$- kappa\_DNA * {C}^{″} + lambda\_DNA * ( ( C - 1 ) + beta * cos ( 2 * pi * r / P ) ) = 0$$

where C″=d\hat{ }2C/dr\hat{ }2. This linearized equation expresses the balance among the three principal effects of the disclosed model: resistance to abrupt spatial variation through kappa_DNA, restoration toward the paired state C=1 through lambda_DNA, and bounded helical forcing through the beta*cos(2*pi*r/P) term. The equation is therefore the direct operational statement of how the disclosed DNA stability formulation acts on the local pairing field over the modeled duplex.

The signs and structure of this equation are physically meaningful. The term involving C″ arises from the kinetic term and opposes rapid curvature or abrupt spatial change in the field, thereby enforcing smoothness of the solution along the duplex. The term lambda_DNA*(C−1) acts as a restoring contribution drawing the field toward the stable paired condition C=1. The term lambda_DNA*beta*cos(2*pi*r/P) introduces a pitch-dependent periodic forcing whose period is set by the helical pitch P. Because beta is bounded and the pairing-well contribution remains dominant, the disclosed equation describes a stable duplex regime in which helical periodicity modulates, but does not overwhelm, the underlying preference for local pairing persistence.

The field C(r) is evaluated over the helical domain extending from the first end boundary (270) to the second end boundary (280), and in disclosed embodiments the boundary conditions are taken as

$$C ( 0 ) = C ( L ) = 1$$

These boundary conditions represent a modeled DNA segment whose ends are fixed or constrained to a paired state. Such boundary conditions are useful because they provide a well-posed finite-domain problem for the disclosed stability analysis and reflect a physically meaningful embodiment in which the duplex ends are not treated as freely opening during the principal stability calculation. Other technically suitable boundary conditions may be used in alternative embodiments, but the paired-end condition C(0)=C(L)=1 is the presently disclosed anchor embodiment and provides a clear basis for solving the governing equation over the finite DNA length L.

The helical phase term cos(2*pi*r/P) is evaluated over the same domain and imposes a repeating positional dependence with period P. As a result, the governing equation is not merely a generic second-order relaxation equation, but a pitch-forced field equation defined on a finite helical interval. This is significant because it ties the solution directly to DNA geometry. A change in pitch P changes the spatial frequency of the forcing term, while a change in lambda_DNA changes the strength of the restoring pairing tendency, and a change in kappa_DNA changes the stiffness against spatial curvature. The disclosed Euler-Lagrange equation therefore provides a compact and explicit mechanism by which sequence stability, helical geometry, and first-principles parameter derivation jointly determine the predicted structural behavior of the duplex.

Where the optional groove-sensitive correction is included, the potential derivative may contain an additional term associated with gamma*(1−G/G0)\hat{ }2*C\hat{ }2. In such embodiments, the full Euler-Lagrange equation may include a groove-related contribution proportional to gamma*(1−G/G0)\hat{ }2*C. However, because the present disclosure treats the groove term as optional and typically secondary in magnitude, the core linearized governing equation stated above remains the principal expression for understanding and implementing the invention. This keeps the central operative path focused on the stiffness term, the sequence-scaled pairing term, and the bounded pitch-dependent forcing term, while still preserving support for groove-sensitive refinements in narrower embodiments.

From a computational standpoint, the disclosed Euler-Lagrange equation may be solved analytically in linearized form, approximately solved under perturbative assumptions, or numerically solved as a boundary-value problem over the interval r in [0,L]. The particular solution method is not the invention; rather, the invention lies in the disclosed DNA Lagrangian density and the resulting governing field equation derived from it. Once this equation is obtained, the solver module (180) of the DNA structural stability prediction system (100) may determine a stable field profile C(r), from which amplitudes, stability metrics, and structural decisions can be computed.

Accordingly, the Euler-Lagrange condition provides the formal variational mechanism by which the DNA Lagrangian density (310) is converted into a concrete governing equation for the local base-pairing probability field. The linearized equation −kappa_DNA*C″+lambda_DNA*((C−1) +beta*cos(2*pi*r/P))=0, evaluated over the helical domain of FIG. 2 and based on the component architecture of FIG. 3, defines the principal field equation used in the present disclosure to predict DNA structural stability. This equation establishes the direct transition from the parameterized Lagrangian model to the solvable stability profile described below.

In the disclosed linearized regime, the Euler-Lagrange equation admits a stable profile for the local base-pairing probability field that may be expressed in closed or approximately closed form as a baseline paired state plus a bounded periodic deviation. In particular, the local base-pairing probability field may be written as

$$C ( r ) = 1 - A * cos ( 2 * pi * r / P ) + epsilon\_decay ( r )$$

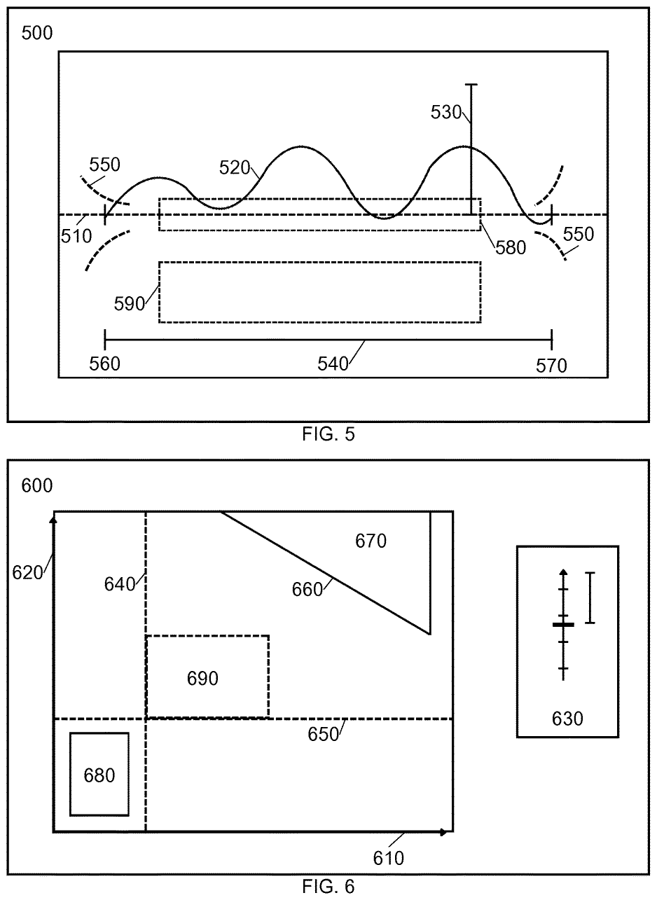

as illustrated by the representative stability profile (500) of FIG. 5. In this expression, the term 1 corresponds to the baseline stability level (510), the oscillatory component defines the stability profile (520) along the helical coordinate axis (540), the quantity A corresponds to the periodic amplitude (530), and the term epsilon_decay(r) corresponds to an end-effect correction (550) associated with the finite interval and the imposed boundary conditions at the first boundary condition indicator (560) and the second boundary condition indicator (570). This closed-form or linearized profile is the principal disclosed solution form for understanding how the DNA Lagrangian density produces a concrete structural stability prediction.

The expression C(r)=1−A*cos(2*pi*r/P)+epsilon_decay(r) follows naturally from the structure of the governing equation-kappa_DNA*C″+lambda_DNA*((C−1)+beta*cos(2*pi*r/P))=0. The term 1 is the preferred local paired state enforced by the pairing-well contribution of the potential. The cosine term arises from the helical phase forcing cos(2*pi*r/P), whose period is set by the helical pitch P. The amplitude A determines the magnitude of the periodic deviation away from the fully paired baseline. The end-effect term epsilon_decay(r) accounts for the fact that the DNA segment is modeled on a finite interval r in [0,L] with constrained ends rather than on an infinite or perfectly periodic domain. Thus, the disclosed profile has a direct physical interpretation: a predominantly paired duplex exhibiting a bounded, pitch-linked modulation in local stability, together with localized corrections near the boundaries.

In disclosed embodiments, the periodic amplitude is given by

$$A = beta * lambda\_DNA / ( kappa\_DNA * ( 2 * pi / P ) \hat{} 2 + lambda\_DNA )$$

This expression shows that the magnitude of the helical modulation is jointly determined by the bounded periodic forcing coefficient beta, the sequence-scaled potential coefficient lambda_DNA, the stiffness parameter kappa_DNA, and the helical pitch P. The numerator beta*lambda_DNA reflects that the amplitude increases with stronger periodic forcing and stronger local pairing-energy scaling. The denominator kappa_DNA*(2*pi/P)\hat{ }2+lambda_DNA reflects the combined resistance to deviation supplied by the stiffness contribution and the restoring pairing-well contribution. Accordingly, the amplitude A is not arbitrary, but is determined by a balance between the helical forcing tendency and the stabilizing influences of gradient rigidity and local pairing preference.

The amplitude expression has several important properties. First, for positive kappa_DNA and positive lambda_DNA, the denominator is strictly positive. Second, because beta is bounded in the disclosed embodiments, A is likewise bounded. Third, in the disclosed stable regime, A<beta, since the denominator contains lambda_DNA plus the additional non-negative stiffness term kappa_DNA*(2*pi/P)\hat{ }2. This inequality is significant because it shows that the structural response of the duplex is smaller than the raw forcing coefficient itself. In other words, the DNA stability profile does not simply mirror the periodic forcing term at full strength; instead, the stiffness and pairing-well structure of the model attenuate the helical modulation. This is consistent with the intended physical interpretation that a structurally stable DNA duplex remains close to the paired baseline and exhibits only limited periodic deviation.

The representative stability profile (500) shown in FIG. 5 therefore captures two distinct stability regimes. A stable operating region (580) corresponds to portions of the profile in which the local base-pairing probability field (520) remains close to the baseline stability level (510), indicating strong persistence of the paired state. A reduced-stability region (590) corresponds to portions of the profile where the oscillatory deviation reaches its local extrema and the field departs further from the baseline. Even in these reduced-stability portions, however, the disclosed bounded amplitude keeps the field within a controlled range for structurally acceptable sequences and geometries. Thus, FIG. 5 is not intended to depict catastrophic strand opening, but rather a bounded modulation around a principally stable duplex condition.

The end-effect correction epsilon_decay(r) represents the influence of the finite domain and the imposed boundary conditions C(0)=C(L)=1. In the interior of a sufficiently long sequence, the oscillatory component may dominate the observed profile and the end-effect correction may become small. Near the first end boundary (270) and the second end boundary (280), however, the end-effect correction may become more pronounced so that the field remains consistent with the imposed boundary conditions. The first boundary condition indicator (560) and second boundary condition indicator (570) shown in FIG. 5 therefore provide visual support for the fact that the profile is not purely an unconstrained cosine, but a finite-domain solution modified near the ends. In some embodiments, epsilon_decay(r) may decay exponentially or otherwise decrease with distance from a boundary, while in other embodiments it may be represented implicitly through a numerical boundary-value solution rather than through a separately parameterized closed-form expression.

The disclosed linearized profile is especially useful because it provides a compact analytic bridge between the governing field equation and the downstream stability metrics. Once C(r) is expressed in the form 1−A*cos(2*pi*r/P)+epsilon_decay(r), quantities such as deviation from baseline, structural stability integrals, and acceptance thresholds can be computed directly or approximately from the amplitude A and the residual end-effect behavior. As a result, the profile form is not merely descriptive; it is operationally useful for ranking candidate constructs, determining whether a given sequence-and-geometry combination lies in a stable regime, and supporting subsequent acceptance criteria based on bounded deviation.

The closed-form or linearized profile also makes clear how each model parameter influences the observed stability behavior. Increasing kappa_DNA increases the denominator of the amplitude expression and therefore reduces A, corresponding to a stiffer duplex with greater resistance to spatial modulation. Increasing lambda_DNA strengthens the restoring pairing tendency and also contributes to the denominator, although it also appears in the numerator, so the resulting effect reflects the balance between stronger local pairing and stronger coupling of the periodic forcing into the local potential. Changing the pitch P changes the spatial frequency 2*pi/P and therefore changes both the shape of the oscillatory component and the stiffness-weighted term in the denominator. Increasing beta increases the amplitude directly, but only within the bounded stable regime imposed by the disclosed formulation. The closed-form profile therefore provides a direct interpretive map from parameter selection to predicted structural behavior.

Accordingly, the expression C(r)=1−A*cos(2*pi*r/P)+epsilon_decay(r), together with A=beta*lambda_DNA/(kappa_DNA*(2*pi/P)\hat{ }2+lambda_DNA), defines the principal disclosed linearized stability profile for the local base-pairing probability field. As shown in FIG. 5, this profile consists of a baseline paired state, a bounded helical modulation, and a finite-domain end correction, thereby providing a physically interpretable and computationally useful description of DNA structural stability under the disclosed Lagrangian-density formulation. This stability profile serves as the immediate basis for the stability metrics and acceptance logic described below.

FIG. 4 illustrates one disclosed implementation pathway for carrying out the present invention through a DNA stability prediction workflow (400). This workflow provides a concrete computational embodiment in which sequence-related and geometry-related inputs are received, model parameters are derived, a DNA Lagrangian density is constructed, a boundary-value problem is solved, and one or more structural-stability outputs are generated and evaluated. The workflow is specifically useful as an enablement anchor because it supplies an operational sequence of acts by which a user, processor, or computational system may apply the disclosed formulation to an actual candidate DNA construct.

In a disclosed embodiment, the workflow begins by receiving sequence-related input at a receive sequence-related input step (410) and geometry-related input at a receive geometry-related input step (420). The sequence-related input may include a sequence stability metric S_stab, a number of base pairs n_bp, a sequence length L, or any suitable sequence-derived quantity from which those values may be obtained. The geometry-related input may include a helical pitch P, a groove ratio G, and one or more RS-derived parameter values including E_coh, E_bp, X_DNA, and, in some embodiments, a reference pitch P0. In this way, the disclosed workflow operates on a candidate DNA construct represented as a coupled sequence-and-shape object rather than as a purely sequence-only input.

At a derive model parameters step (430), the disclosed embodiment computes the principal coefficients used in the DNA Lagrangian density. In particular, the stiffness parameter may be computed as kappa_DNA=E_coh*X_DNA. An effective base-pair axial scale may be computed as a_bp=X_DNA/4. The sequence-scaled potential coefficient may then be computed as lambda_DNA=(E_bp/a_bp)*S_stab. The periodic modulation coefficient may be computed from the Recognition Science-based ratio beta=E_coh*P0/(hbar*c), or, in another embodiment, a bounded coefficient may be selected such that 0<beta<1. In some implementations, the workflow may also select one or more operational criteria at this stage, including an amplitude threshold delta_A, a minimum S_stab threshold, or one or more thresholds on derived stability metrics such as M_struct and M_melt.

At a construct Lagrangian step (440), the workflow uses the computed coefficients to form the disclosed DNA Lagrangian density over a modeled helical domain. In disclosed embodiments, the modeled domain is defined by setting r in [0,L], and the helical phase term is computed as cos(2*pi*r/P). The workflow also imposes the disclosed boundary conditions C(0)=C(L)=1, thereby defining a finite-domain problem with paired end conditions. In this manner, the timing or phase computation is not left abstract. Rather, the workflow explicitly defines the helical interval, computes the pitch-linked periodic phase term over that interval, and constrains the field at the boundaries before solution of the governing equation. If DNARP-related language is used in connection with this embodiment, any pattern-measurement relation may be introduced only as contextual support for the inputs and not as a separate claimed invention.

At a solve Euler-Lagrange equation step (450), the workflow solves the disclosed boundary-value problem associated with the DNA Lagrangian density. In one embodiment, the governing equation is the linearized field equation-kappa_DNA*C″+lambda_DNA*((C−1)+beta *cos(2*pi*r/P))=0 subject to the boundary conditions C(0)=C(L)=1. The solution may be obtained analytically in a linearized regime, approximately under perturbative assumptions, or numerically using a boundary-value solver. In one disclosed computational implementation, the boundary-value problem is solved using SciPy solve_bvp, although other technically suitable solvers may be used. The solve step therefore constitutes the action by which the input parameters are transformed into a concrete local base-pairing probability field over the finite DNA domain.

At a generate stability profile step (460), the workflow produces the local base-pairing probability field C(r) over the interval r in [0,L]. In a disclosed linearized embodiment, the resulting profile may take the form C(r)=1−A*cos(2*pi*r/P)+epsilon_decay(r). The workflow may determine the amplitude A directly from the solution or from the amplitude relation A=beta*lambda_DNA/(kappa_DNA*(2*pi/P)\hat{ }2+lambda_DNA). The generated profile therefore provides a position-dependent representation of local structural stability along the DNA duplex and serves as the basis for subsequent metric computation and decision-making.

At a compute stability metric step (470), the workflow computes one or more quantitative measures from the generated profile. In disclosed embodiments, these measures include the periodic amplitude A, the structural stability metric M_struct, and the melting-barrier metric M_melt. The structural stability metric may be computed as M_struct=integral_0\hat{ }L (1−C(r))\hat{ }2 dr, and the melting-barrier metric may be computed as M_melt=lambda_DNA(S_stab)*L/2. In optional embodiments, the workflow may also compute a damage-resistance metric M_damage. The act of computing these metrics converts the field solution into compact values suitable for design validation, ranking, or acceptance analysis.

At a stability decision step (480), the workflow applies one or more operational decision rules to determine whether the candidate construct is acceptable for a target use. In one disclosed embodiment, the construct is classified as stable if |A|<=0.05 and unstable if |A|<0.05. In additional or alternative embodiments, the workflow may require that S_stab>=3, that P in

Angstrom, and that G in [1.5,1.7]. In still further embodiments, the workflow may require satisfaction of application-specific thresholds on M_struct, M_melt, or bounds on C(r). These criteria may be applied individually or in combination. For example, a candidate sequence may be accepted only if it satisfies the amplitude threshold and the sequence-stability threshold, or only if it also remains within a target pitch range and exhibits a minimum melting barrier. Thus, the workflow includes an explicit verification metric and decision rule rather than stopping at mere profile generation.

At an output result step (490), the workflow outputs one or more results responsive to the applied decision logic. The output may include the field profile C(r), the computed amplitude A, the structural stability metric M_struct, the melting-barrier metric M_melt, an optional damage-resistance metric M_damage, and a corresponding stable, unstable, accept, reject, or rank-ordered determination. In a DNARP-related implementation, an unstable result may cause a candidate construct to be rejected or halted within a simulator or optimization loop, whereas a stable result may permit the construct to proceed for further evaluation, ranking, synthesis planning, archival selection, or therapeutic validation.

The workflow of FIG. 4 may be implemented by the DNA structural stability prediction system (100) of FIG. 1. For example, the receive sequence-related input step (410) and receive geometry-related input step (420) may be carried out through the input interface (110), the derive model parameters step (430) may be carried out through the parameter derivation module (160), the construct Lagrangian step (440) may be carried out through the Lagrangian construction module (170), the solve Euler-Lagrange equation step (450) may be carried out through the solver module (180), and the generate stability profile step (460), compute stability metric step (470), stability decision step (480), and output result step (490) may be carried out through the output module (190) and associated computational logic. In this way, the present disclosure provides a fully implementable operational path from input acquisition through decision output.

Accordingly, the DNA stability prediction workflow (400) provides one concrete way to implement the present invention. It identifies the incoming data, specifies the named method and operational parameters, defines the helical-domain and phase computation, states the action taken to solve the governing equation and generate a stability profile, and applies one or more quantitative verification metrics and decision rules to produce an actionable structural-stability result. This workflow therefore supplies a complete operational embodiment by which the disclosed DNA Lagrangian-density formulation may be used in practice to evaluate candidate DNA constructs.

Following generation of the local base-pairing probability field C(r), the present disclosure derives one or more quantitative stability metrics that convert the solved field profile into compact measures suitable for structural assessment, candidate ranking, and acceptance analysis. These metrics are useful because the field profile itself provides a position-dependent description of stability, whereas many practical design and evaluation workflows benefit from one or more scalar values that summarize structural persistence, resistance to strand separation, or robustness against destabilization. In disclosed embodiments, the principal metrics include a structural stability metric M_struct and a melting-barrier metric M_melt, with an optional damage-resistance metric M_damage in narrower embodiments.

A first disclosed metric is the structural stability metric M_struct, which may be written as

$$M\_struct = integral\_ 0 \hat{} L ( 1 - C ( r ) ) \hat{} 2 dr$$

This quantity measures the accumulated deviation of the local base-pairing probability field from the idealized fully paired baseline C(r)=1 over the modeled DNA domain. Because the integrand (1-C(r))\hat{ }2 is non-negative for all r, the metric M_struct is likewise non-negative. The quantity M_struct therefore provides a direct measure of how far the predicted stability profile departs from the perfectly paired state. When the field remains very close to C(r)=1 throughout the interval, M_struct is small, indicating strong structural persistence. When the field departs more substantially from the fully paired condition, M_struct increases, indicating reduced structural stability. The metric therefore serves as a compact integral measure of deviation from the stable baseline.

The metric M_struct also has a useful interpretive property in the disclosed formulation: it vanishes only when the field is identically equal to the paired baseline over the modeled interval. More specifically, M_struct=0 if and only if C(r)=1 throughout the interval r in [0,L], because only then does the non-negative integrand vanish everywhere. In practical embodiments, exact vanishing may not be expected once helical modulation and finite-domain effects are included, but the closer M_struct is to zero, the closer the modeled duplex remains to the idealized paired condition. This makes M_struct particularly useful as an acceptance and ranking metric, since smaller values correspond naturally to stronger predicted structural stability.

In the disclosed linearized regime, M_struct may be approximated from the periodic amplitude A. Using the representative stability profile of FIG. 5, where C(r)=1−A*cos(2*pi*r/P)+epsilon_decay(r), and neglecting or averaging small end-effect contributions, the dominant oscillatory component gives

$$M\_struct approx A \hat{} 2 * L / 2$$

This approximation follows from integrating the square of the cosine-driven deviation over the interval and using the average value of cos\hat{ }2 over a period. The approximation is useful because it ties the scalar structural stability metric directly to the amplitude of the helical modulation. Thus, in the stable regime where A is small, M_struct is also small, reinforcing the interpretation that bounded periodic deviation corresponds to strong structural persistence.

A second disclosed metric is the melting-barrier metric M_melt, which may be written as

$$M\_melt = lambda\_DNA ( S\_stab ) * L / 2$$

This quantity provides an energy-related measure associated with full strand separation or large-scale loss of pairing persistence over the modeled interval. Whereas M_struct measures deviation of the solved field from the idealized paired profile, M_melt reflects the effective barrier associated with separating the duplex against the local pairing tendency encoded by lambda_DNA. Because lambda_DNA is positive in disclosed embodiments and L is positive for any nonzero modeled sequence length, M_melt is likewise positive. The metric therefore serves as a compact measure of resistance to melting or global destabilization, with larger values corresponding to stronger resistance to strand separation.

The disclosed form of M_melt is also monotone with respect to the sequence stability metric S_stab. Since lambda_DNA(S_stab)=(E_bp/a_bp)*S_stab, increasing S_stab increases lambda_DNA, which in turn increases M_melt. This is consistent with the intended physical meaning of the formulation: sequences having greater effective pairing strength should exhibit a larger barrier to melting, while sequences having weaker effective pairing strength should exhibit a smaller barrier. The metric M_melt therefore provides a useful scalar indicator of global duplex resistance to strand opening and is especially suitable for use in threshold-based acceptance logic, screening, and ranking among candidate sequences.

In some embodiments, the present disclosure may further include a calibrated or mapped melting-temperature-related quantity derived from M_melt or from the underlying sequence-scaled potential structure. Where such an embodiment is used, any relation to a melting temperature T_m is preferably treated as an additional calibrated output rather than as a universal direct identity. This drafting posture preserves flexibility while recognizing that the present invention is directed principally to structural stability prediction through the disclosed Lagrangian-density framework, not to an exclusive or exhaustive empirical temperature model. Accordingly, a T_m-related output may be provided in some implementations, but the core operative metrics remain M_struct and M_melt.

In narrower embodiments, the present disclosure may also employ an optional damage-resistance metric M_damage. This metric may characterize resistance to perturbation, degradation, or externally induced destabilization as a function of the modeled stability state. For example, M_damage may be defined from one or more combinations of the amplitude A, the profile C(r), the structural stability metric M_struct, the melting-barrier metric M_melt, or other derived quantities that reflect robustness of the duplex under expected use conditions. The optional character of M_damage is intentional. It preserves support for embodiments in which an additional robustness metric is useful, while keeping the core invention centered on the principal structural and melting-barrier measures.

These metrics are operationally important because they allow the local field solution to be converted into decision-ready values. The stability profile C(r) provides the detailed positional behavior of the duplex, but M_struct, M_melt, and, where used, M_damage provide compact measures that can be directly compared to thresholds, used to rank candidate constructs, or employed in an optimization or validation loop. For example, a construct having a small M_struct and a large M_melt may be classified as highly stable, while a construct having a larger M_struct and a smaller M_melt may be rejected, deprioritized, or subjected to redesign. In this way, the metrics provide a direct link between the field-theoretic solution and practical design decision-making.

The disclosed metrics also preserve physical interpretability. M_struct reflects deviation from the ideal paired baseline and therefore captures how much the solved duplex profile departs from local structural persistence. M_melt reflects the integrated strength of the pairing contribution over the modeled interval and therefore captures the effective barrier to global strand separation. M_damage, where used, captures an additional notion of robustness or resistance to destabilizing influence. Because these metrics are derived from the disclosed field profile and the disclosed parameter structure, they are not disconnected reporting values; instead, they remain tightly tied to the first-principles DNA Lagrangian-density formulation that generated the profile in the first place.

Accordingly, the present disclosure employs M_struct and M_melt as the principal quantitative stability metrics derived from the solved local base-pairing probability field, with optional support for M_damage and calibrated temperature-related outputs in narrower embodiments. These metrics convert the position-dependent field solution into compact, physically meaningful quantities that can be used for acceptance analysis, ranking, optimization, and downstream application-specific decision-making. In this manner, the stability metrics provide the immediate analytical bridge between the closed-form or linearized stability profile and the operational acceptance criteria described below.

In disclosed embodiments, the present invention may employ one or more operational envelopes and acceptance criteria to determine whether a candidate DNA construct falls within a structurally acceptable regime for a target use. These operational criteria are optional in the sense that the core Lagrangian-density formulation is capable of generating a stability profile and associated metrics without any particular threshold set being mandatory. However, in practical implementations, acceptance criteria are useful because they convert the solved field profile and derived metrics into an actionable design decision. The present disclosure therefore provides support for threshold-based, range-based, and combined acceptance logic that may be applied to the quantities generated by the disclosed DNA structural stability framework.

One disclosed class of acceptance criteria is based on the periodic amplitude A of the local base-pairing probability field. Because A measures the magnitude of the helical modulation away from the baseline paired condition, it provides a direct scalar indicator of how strongly the modeled duplex departs from local structural persistence. In one embodiment, an amplitude threshold delta_A is selected, and a candidate construct is accepted only if |A|<=delta_A. A disclosed illustrative value is delta_A=0.05. Under this criterion, a construct whose predicted profile remains within five hundredths of the normalized pairing baseline in amplitude is considered structurally acceptable, whereas a construct exhibiting a larger oscillatory deviation may be rejected, deprioritized, or flagged for redesign. This amplitude-based criterion is useful because it ties acceptance directly to the bounded helical modulation predicted by the disclosed formulation.

A further disclosed class of acceptance criteria is based on the sequence stability metric S_stab. Because S_stab controls the depth of the effective pairing well through lambda_DNA(S_stab), it provides a direct measure of sequence-derived pairing strength within the disclosed model. In one embodiment, a candidate construct is required to satisfy S_stab>=3. This threshold is illustrative of a regime in which the sequence-derived pairing contribution is sufficiently strong to support a stable duplex under the disclosed formulation. Constructs below that threshold may be treated as insufficiently stable for the intended application, particularly where the target use requires stronger persistence against local destabilization or melting. This criterion is useful because it enables early filtering of candidates at the sequence-derived stability level before or in conjunction with full profile-based evaluation.

The present disclosure also provides geometry-based operational envelopes. In one embodiment, the helical pitch P is required to lie within a pitch range P in [30,40] Angstrom. This range is illustrative of a structurally admissible regime for the disclosed pitch-dependent helical modulation term. If the helical pitch falls substantially outside this interval, the spatial periodicity imposed by the phase term cos(2*pi*r/P) may no longer correspond to a preferred structural regime for the DNA duplex being modeled. Accordingly, a candidate construct may be accepted only if its pitch falls within the disclosed operational window, or may be ranked more favorably when its pitch lies closer to a preferred reference value within that interval. This pitch criterion therefore provides a direct way to use the geometric input model in downstream structural acceptability logic.

In addition to pitch, the groove ratio G may also be used as an operational acceptance quantity. In one embodiment, a candidate construct is evaluated within a groove-ratio range G in [1.5,1.7]. This range corresponds to a disclosed window in which groove geometry remains consistent with the intended helical structural regime and, in embodiments using the optional groove-correction term, remains sufficiently close to the reference groove relation that the groove-sensitive correction remains limited in magnitude. A construct falling outside that range may be classified as less preferred, geometrically non-ideal, or unacceptable for a given implementation. Thus, even though the groove-sensitive correction is optional, groove ratio may still be used as an acceptance-screening quantity in embodiments where geometric conformity is relevant to downstream application requirements.

As illustrated in FIG. 6, one or more acceptance-region diagrams (600) may be defined using a sequence stability axis (610), a helical pitch axis (620), and, in some embodiments, a groove-ratio axis (630). Within such a diagram, a structural stability metric threshold (640), a melting barrier metric threshold (650), and an amplitude threshold (660) may be used to partition the parameter space into an accepted design region (670), a rejected design region (680), and a transition region (690). The accepted design region (670) corresponds to combinations of sequence-derived stability and geometry that satisfy the selected operational criteria. The rejected design region (680) corresponds to combinations that fail one or more criteria. The transition region (690) corresponds to combinations near one or more boundaries, where a candidate may require additional analysis, narrower tolerance selection, or context-specific ranking. In this way, the disclosed acceptance criteria may be visualized not merely as independent scalar thresholds, but as an operational design space within which DNA candidates can be screened and classified.

Acceptance logic may also be based directly on the disclosed stability metrics. In one embodiment, a candidate construct is accepted only if M_struct is less than or equal to a selected structural threshold and M_melt is greater than or equal to a selected melting-barrier threshold. Smaller M_struct values indicate reduced deviation from the paired baseline, while larger M_melt values indicate stronger resistance to strand separation. A candidate satisfying both criteria may therefore be classified as structurally robust under the disclosed formulation. In optional embodiments, an additional threshold may be applied to M_damage, where used, in order to preserve support for robustness-based or perturbation-resistance screening. These metric-based criteria are particularly useful in optimization workflows because they allow multiple candidates to be compared using common scalar quantities derived from the same field-theoretic foundation.

The disclosed acceptance criteria may be applied individually or in combination. For example, an implementation may require only |A|<=0.05 for a binary stable/unstable classification. Another implementation may require both |A|<=0.05 and S_stab>=3. A further implementation may require satisfaction of the amplitude criterion, the sequence-stability criterion, the pitch envelope P in [30,40] Angstrom, and the groove-ratio envelope G in [1.5,1.7]. Still further embodiments may evaluate candidates using a weighted or hierarchical logic in which failure of a primary threshold causes immediate rejection, while satisfaction of all primary thresholds and selected secondary thresholds causes acceptance or ranking improvement. The present disclosure therefore supports both simple threshold logic and more elaborate multi-criterion acceptance frameworks.

In some embodiments, a reference pitch quantity P0, including embodiments in which P0 approx 35.6 Angstrom, may also be used as a comparison or calibration quantity in determining whether a candidate pitch remains close enough to a preferred helical periodicity. Likewise, disclosed bounded values such as beta_base approx 0.016 and beta_eff approx 0.052 may be used to define acceptable periodic-forcing regimes in which beta remains a modulation factor rather than a dominant destabilizing term. These values are illustrative of acceptable operating regimes for the disclosed formulation and may be used to constrain model selection, profile interpretation, or acceptance analysis where it is desirable to ensure that the system remains within the intended stable-duplex modeling regime.

The practical function of these operational envelopes is to convert the solved Lagrangian-based stability analysis into a technical decision suitable for design validation and downstream use. A construct satisfying the selected acceptance criteria may be passed forward for additional evaluation, synthesis planning, archival selection, therapeutic screening, or other application-specific processing. A construct failing one or more criteria may be rejected, deprioritized, or returned for redesign. In some embodiments, acceptance or rejection may be reported through the application-specific output system (1000) described elsewhere in the present disclosure, including via a stability score display (1060), an acceptance indicator (1070), a rejection indicator (1080), or a report generation module (1090). Thus, the acceptance criteria do not merely annotate the results of the formulation; they provide the operational decision layer by which the formulation is used in practice.

Accordingly, the present disclosure provides optional operational envelopes and acceptance criteria based on amplitude, sequence stability, pitch, groove ratio, and one or more derived stability metrics. These criteria may define accepted, rejected, and transition regions within the design space and may be applied in binary, threshold-based, multi-criterion, or ranking-oriented decision frameworks. In this manner, the disclosed acceptance logic provides a practical bridge from the solved stability profile and derived metrics to an actionable structural assessment of candidate DNA constructs.

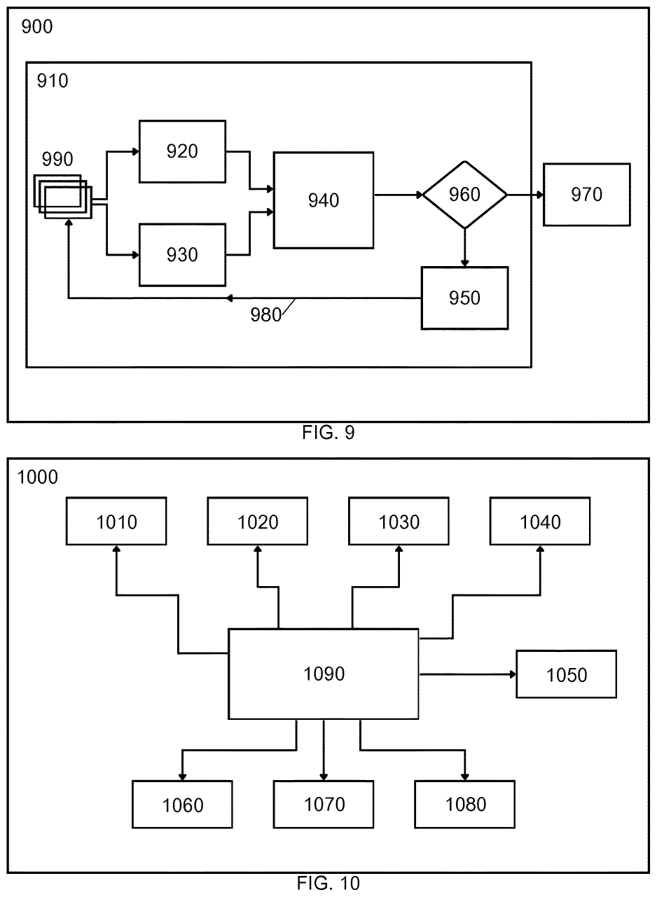

In some embodiments, the disclosed DNA structural stability formulation is integrated into a DNARP workflow as a stability-analysis and validation component operating on candidate sequence-and-shape objects. As illustrated in FIG. 9, a DNARP integration system (900) may include a DNARP pipeline (910) having a sequence generation module (920), a shape generation module (930), a DNA stability evaluation engine (940), an optimization loop (950), an acceptance filter (960), a validated construct output (970), a feedback path (980), and a design candidate set (990). Within this arrangement, the present invention functions as the DNA stability evaluation engine (940) that receives candidate constructs generated upstream, evaluates those constructs according to the disclosed Lagrangian-density formulation, and returns a stability-related result suitable for filtering, ranking, or iterative redesign.

A DNARP candidate may be represented in terms of sequence and shape quantities, including a sequence component S and a shape component H, and may in some embodiments be described using notation of the form D=(S, H, E). In this context, the present invention is particularly suited to operate on the sequence-related and geometry-related aspects of the candidate object. The sequence generation module (920) may generate, select, mutate, or otherwise provide candidate DNA sequences, while the shape generation module (930) may provide corresponding geometric quantities such as helical pitch P, groove ratio G, or related structural descriptors. The DNA stability evaluation engine (940) then evaluates whether the resulting candidate is structurally acceptable under the disclosed formulation by computing the local base-pairing probability field C(r), one or more derived metrics, and an associated stability decision.

The integration shown in FIG. 9 is useful because the present invention does not require DNARP to be redefined or replaced. Rather, it supplies a stability-analysis layer that may be inserted into an existing candidate-generation and optimization framework. In disclosed embodiments, the design candidate set (990) may include one or more candidate sequences, one or more candidate helical geometries, or one or more candidate pairings of sequence and shape. These candidates may be generated de novo, selected from a library, produced through mutation or recombination, or generated by an upstream computational design routine. The sequence generation module (920) and shape generation module (930) may therefore operate independently or cooperatively to provide candidate inputs for stability analysis.

Once a candidate is supplied, the DNA stability evaluation engine (940) may determine a sequence stability metric S_stab, a sequence length L, a helical pitch P, a groove ratio G, and one or more first-principles parameters including E_coh, E_bp, X_DNA, and, where used, P0. From these quantities, the engine may derive kappa_DNA, lambda_DNA, and beta, construct the DNA Lagrangian density, solve the corresponding Euler-Lagrange equation over the interval r in [0,L], and generate the local base-pairing probability field C(r). The engine may then compute one or more stability metrics including A, M_struct, and M_melt, and may optionally compute M_damage or a related robustness quantity. In this manner, the disclosed invention operates within the DNARP environment as a technically concrete filter on structural viability rather than as a generic advisory layer.

The acceptance filter (960) may apply one or more of the disclosed acceptance criteria to the output of the DNA stability evaluation engine (940). For example, a candidate may be passed by the acceptance filter (960) only if |A|<=0.05, S_stab>=3, P in [30,40] Angstrom, G in [1.5,1.7], M_struct is below a selected threshold, and M_melt is above a selected threshold. In simpler embodiments, the acceptance filter (960) may apply only one or two of these criteria. In more elaborate embodiments, the filter may apply weighted or hierarchical decision logic, such as rejecting a candidate immediately if a primary amplitude criterion fails, or assigning a rank score if all primary criteria are satisfied but one or more secondary criteria are near a transition boundary. The acceptance filter (960) therefore provides the direct mechanism by which the solved stability profile and derived metrics are converted into DNARP-usable design decisions.

Candidates satisfying the applied criteria may be forwarded to the validated construct output (970). A validated construct may be a sequence-shape pair determined to fall within an accepted design region under the disclosed formulation, a ranked candidate selected for further downstream evaluation, or a candidate approved for additional design processing, synthesis planning, archival storage assessment, or therapeutic screening. The validated construct output (970) may therefore provide a simple binary pass result, a rank-ordered list, or a richer structured output including the underlying profile C(r) and associated stability metrics. In some embodiments, only the highest-ranked candidates are retained for further use, while other candidates are discarded or recycled into the optimization process.

Candidates that do not satisfy the applied acceptance logic may be routed through the optimization loop (950) and the feedback path (980). In this way, the present invention not only evaluates candidate DNA constructs, but also informs iterative improvement of those constructs within a DNARP environment. For example, if a candidate fails because M_struct is too large or because |A|exceeds a permissible threshold, the upstream system may alter sequence composition, modify helical pitch, adjust groove-related geometry, or otherwise generate a revised candidate for re-evaluation. The feedback path (980) therefore allows the disclosed formulation to operate as part of a closed-loop design framework in which structural-stability prediction directly influences subsequent candidate generation.

This integration posture is particularly useful because it preserves the modularity of the present invention. The DNA stability evaluation engine (940) may be implemented as a standalone module, a callable service, a solver-backed function, or an embedded subsystem within a larger design platform. In some embodiments, the engine may be implemented using the DNA structural stability prediction system (100) described elsewhere in the present disclosure, such that the input interface (110), parameter derivation module (160), Lagrangian construction module (170), solver module (180), and output module (190) collectively instantiate the DNA stability evaluation engine (940). In other embodiments, only part of system (100) is embedded directly in the DNARP pipeline (910), while the remainder is implemented remotely or through shared computing resources. Accordingly, FIG. 9 is intended to show functional integration rather than impose a single architectural constraint.

The present invention may also support design-stage validation of candidate geometry before physical realization. Because the shape generation module (930) may produce candidate pitch or groove parameters along with sequence content, the DNA stability evaluation engine (940) may reject geometrically noncompliant candidates even when sequence-derived stability alone appears adequate. Conversely, the engine may identify candidates whose sequence composition is sufficiently robust to remain structurally acceptable across a range of pitch or groove values. This allows the DNARP integration system (900) to evaluate candidate constructs as coupled sequence-and-shape objects rather than as sequence-only artifacts, thereby improving structural realism within the design pipeline.

In some embodiments, the DNA stability evaluation engine (940) may also be used for comparative or competitive ranking among multiple design candidates. For instance, the design candidate set (990) may include a population of related sequences generated to satisfy a common functional objective, and the present invention may compute A, M_struct, M_melt, or related quantities for each candidate. The optimization loop (950) may then use those quantities to prioritize candidates exhibiting lower predicted structural deviation and greater resistance to strand separation. This ranking mode is fully consistent with the disclosed acceptance framework and provides a practical DNARP-compatible mechanism for selecting preferred constructs among a set of otherwise functionally similar candidates.

Accordingly, the DNARP integration system (900) shown in FIG. 9 demonstrates how the disclosed DNA Lagrangian-density formulation may be used as a sequence-and-shape validation engine within a broader computational design environment. The sequence generation module (920) and shape generation module (930) provide candidate constructs, the DNA stability evaluation engine (940) applies the disclosed first-principles formulation to those constructs, the acceptance filter (960) determines whether they satisfy structural criteria, the validated construct output (970) supplies accepted or ranked candidates, and the optimization loop (950) with feedback path (980) supports iterative redesign where needed. In this manner, the present invention integrates naturally into DNARP workflows as a technically concrete structural-stability assessment component for DNA construct design and validation.

In order to further illustrate operation of the disclosed DNA Lagrangian-density formulation, the present disclosure provides a series of worked examples showing how the formulation may be applied to representative candidate constructs, low-stability rejection cases, comparative output calibration, archival-storage design, geometric envelope validation, and groove-sensitive refinement. These worked examples are illustrative only and are not intended to limit the scope of the invention. In each case, the example follows the same general pattern: identify the relevant inputs, apply the disclosed parameter relations and governing equations, obtain or characterize the resulting stability profile, compute one or more stability metrics, and then interpret the result in terms of structural acceptability, rejection, ranking, or application-specific utility. As described below, FIG. 8 is particularly useful for comparative sequence embodiments, and FIG. 10 is particularly useful for application-specific output embodiments.

In one illustrative embodiment, the disclosed formulation is applied to a SIRT1 DNARP program or comparable candidate construct produced in a DNARP workflow. In this example, the relevant inputs include a sequence stability metric S_stab, a sequence length L, a helical pitch P, and one or more Recognition Science-derived quantities including E_coh, E_bp, X_DNA, and reference pitch P0. The parameter derivation proceeds according to the disclosed relations kappa_DNA=E_coh*X_DNA, a_bp=X_DNA/4, lambda_DNA=(E_bp/a_bp)*S_stab, and beta=E_coh*P0/(hbar*c) or another bounded periodic modulation coefficient satisfying 0<beta<1. In one disclosed illustrative numerical path, kappa_DNA=E_coh*X_DNA=1.224 eV*Angstrom, a_bp=X_DNA/ 4 approx 3.4 Angstrom, and beta=beta_base approx 0.016. For a representative pitch P=32 Angstrom, the helical spatial frequency is k=2*pi/P approx 0.196 Angstrom\hat{ }−1, and the amplitude may be written as A=beta*lambda_DNA/(kappa_DNA*k\hat{ }2 +lambda_DNA), with A<1 beta in the disclosed stable regime. The resulting local base-pairing probability field may therefore be expressed as C(r) approx 1−A*cos(2*pi*r/P), optionally with finite-domain end correction. Because beta is small and the sequence is assumed to satisfy the minimum DNARP stability condition, the resulting profile remains close to the paired baseline and yields a positive melting barrier M_melt=lambda_DNA*L/2. This example therefore illustrates a candidate that passes the disclosed stability analysis and may be reported as a design validation output (1010), a stability score display (1060), and an acceptance indicator (1070) within the application-specific output system (1000) of FIG. 10.

In another illustrative embodiment, the disclosed formulation is applied to a below-threshold sequence. In this example, the controlling input difference is that the sequence stability metric S_stab is small, for example below a minimum threshold such as S_stab<3. Because lambda_DNA(S_stab)=(E_bp/a_bp)*S_stab, a reduction in S_stab directly reduces the depth of the local pairing well. As a result, the melting-barrier metric M_melt=lambda_DNA*L/2 is correspondingly reduced, indicating weaker global resistance to strand separation. Although the periodic modulation amplitude remains bounded by beta, the candidate may nonetheless fail the operational stability criteria because the sequence-derived pairing contribution is insufficient for the intended application. In such an embodiment, the disclosed workflow may reject the candidate directly on the basis of the sequence-stability threshold, or may reject it after confirming that the resulting M_melt falls below a target minimum. This example is useful because it shows that the present formulation does not merely model oscillatory stability behavior, but also supports direct rejection of insufficiently stable candidates based on sequence-driven weakening of the pairing potential. A result of this kind may be reported through a rejection indicator (1080) or a report generation module (1090) within the application-specific output system (1000).

In a further illustrative embodiment, the disclosed formulation is compared to SantaLucia-type nearest-neighbor output or another empirical thermodynamic reference model. In this example, the disclosed DNA Lagrangian density is used to derive intrinsic stability quantities such as M_struct and M_melt from RS-derived constants and the candidate-specific inputs S_stab, P, and G. The comparison emphasizes that the present formulation does not require per-nearest-neighbor fitted parameter tables in order to generate a structural stability profile or a melting-barrier measure. At the same time, the example acknowledges that experimentally measured melting temperature T_m depends not only on a barrier-related energy scale, but also on entropy and assay-specific environmental conditions such as salt concentration, strand concentration, and buffer chemistry. Accordingly, where a predicted experimental T_m output is desired, the present disclosure treats the mapping from M_melt to a T_m-related output as an additional calibrated embodiment rather than as a universal direct identity. This example is useful because it preserves technical support for a comparison to empirical oligomer-melting approaches while maintaining the invention's core identity as a first-principles structural stability formulation. In application-facing embodiments, a result of this type may be reported as a design validation output (1010) or a report generation module (1090) output in FIG. 10.

In another illustrative embodiment, the disclosed formulation is applied to DNA data-storage design, particularly archival-sequence selection. In such an example, the design objective is to select a candidate construct having high structural persistence over long storage intervals, such as a target horizon on the order of hundreds or thousands of years. A representative input set may include a comparatively high sequence stability metric, for example S_stab=30 corresponding to a highly CG-rich sequence, together with a helical pitch selected near a reference pitch such as P=35.6 Angstrom and a groove ratio selected near a reference groove value such as G=1.618. Under the disclosed relation lambda_DNA proportional to S_stab, the relatively large S_stab value yields a deeper pairing well and therefore a larger melting barrier M_melt=lambda_DNA*L/2. Selecting a pitch near the reference pitch and a groove ratio near the reference groove condition further supports a stability profile C(r) that remains close to the paired baseline over the interval of interest. In this example, the resulting candidate may be classified as favorable for archival stability because it exhibits both a low deviation profile and a comparatively large barrier to destabilization. This embodiment maps directly to the archival storage construct output (1030) of the application-specific output system (1000), and may also contribute to a rank-ordered candidate output (1050) when multiple candidate archival sequences are screened. In other embodiments, a candidate that satisfies corresponding structural-stability criteria for therapeutic use may be provided as a therapeutic construct output (1040).

In a further illustrative embodiment, the disclosed formulation is used to validate a pitch constraint such as P in [30,40] Angstrom. In this example, the Lagrangian density is evaluated across multiple pitch values while holding other quantities fixed or within a selected operating envelope, and the resulting stability profiles are examined with respect to one or more operational acceptance criteria. These criteria may include maintaining C(r) within the physical interval [0,1], requiring M_struct to remain below a selected threshold, requiring M_melt to remain above a selected threshold, and optionally requiring |A|<=delta_A. By comparing outcomes across the pitch range, the disclosed method identifies a practical admissible interval centered near the reference pitch P0, thereby supporting the proposition that pitches outside the interval are less preferred or unacceptable for a target application. This example is useful because it demonstrates how the formulation can generate a design-space constraint from the model itself rather than from an externally imposed rule alone. In some embodiments, the resulting pass/fail or ranked pitch assessment may be conveyed through the design validation output (1010), the stability score display (1060), or the acceptance indicator (1070) and rejection indicator (1080) of FIG. 10.

In another illustrative embodiment, the optional groove-correction contribution is evaluated to determine the effect of a non-reference groove ratio. In this example, the potential term includes gamma*(1−G/G0)\hat{ }2*C\hat{ }2, and the groove ratio G is varied relative to reference groove ratio G0. For a representative case in which G=1.70 and G0=phi=1.618, the deviation factor (1−G/G0)\hat{ }2 is small, and the resulting groove-sensitive contribution remains much smaller than the principal sequence-scaled pairing contribution. Thus, within the disclosed groove-ratio window, the profile C(r) and the derived metrics remain dominated by kappa_DNA, lambda_DNA, and beta, while the groove correction acts as a secondary refinement. This example is useful because it demonstrates that the optional groove term can be included without altering the core structure of the invention, and that the groove term may be operationally negligible within a preferred geometric window even though it preserves support for narrower groove-sensitive embodiments. Where a groove-sensitive comparison is reported, the result may again be conveyed through the design validation output (1010) or report generation module (1090) of FIG. 10.

FIG. 8 illustrates a further class of worked example in which the present invention is applied to comparative sequence analysis, such as mutation or variant evaluation. In such an embodiment, a reference sequence input (810) and a variant sequence input (820) are processed by a sequence comparison module (830) and a parameter update module (840) of the mutation comparison workflow (800). The reference and variant candidates may then be evaluated under the disclosed formulation to generate a reference stability profile (850) and a variant stability profile (860), respectively. From these profiles, a differential stability metric (870) may be computed, such as a difference in amplitude, a difference in M_struct, a difference in M_melt, or a composite stability-difference measure derived therefrom. A mutation impact decision (880) may then classify the variant as stability-preserving, stability-reducing, or otherwise relevant to the target design context, and the result may be provided as a mutation comparison output (890). This worked-example format is useful because it extends the invention from single-candidate evaluation to comparative ranking and mutation-aware design. In application-specific output terms, such a result may correspond to a mutation assessment output (1020) in FIG. 10.

These worked examples together demonstrate that the disclosed DNA Lagrangian-density formulation is not limited to a single abstract solution scenario. Rather, it can be used to validate a stable DNARP construct, reject a below-threshold sequence, generate a calibrated comparison to empirical oligomer-melting frameworks, select archival DNA candidates, derive acceptable pitch windows, evaluate groove-related refinements, and compare reference and variant sequences in mutation-analysis workflows. In each case, the same underlying formulation is used: a candidate construct is represented through sequence-and geometry-related inputs, the disclosed coefficients are derived, the DNA Lagrangian density is formed, the local base-pairing probability field is solved or approximated, one or more metrics are computed, and a technical decision or interpretation is produced. These examples therefore illustrate the breadth of practical use of the disclosed formulation while remaining fully anchored to the same operative field-theoretic structure described throughout the specification.

The present disclosure further contemplates alternative embodiments and equivalent implementations of the disclosed DNA structural stability formulation, provided that such embodiments remain anchored to the same core invention: a Lagrangian-density framework acting on a local base-pairing probability field C(r) over a DNA domain and using first-principles-derived parameters to predict structural stability. These alternative embodiments are included to preserve technical flexibility, amendment support, and equivalent-implementation coverage, but they do not alter the fact that the core invention is centered on the disclosed DNA Lagrangian density, the resulting Euler-Lagrange field equation, the derived stability profile, and the associated stability metrics. Accordingly, the alternatives described below are to be understood as optional variants or equivalents of the disclosed formulation rather than separate inventions.

In one class of alternative embodiments, the potential structure may include one or more higher-order harmonic contributions. For example, in addition to the principal periodic term -lambda_DNA*beta*(1−C)*cos(2*pi*r/P), the potential may include a second-harmonic contribution proportional to C\hat{ }2*cos(4*pi*r/P) or another higher-order periodic term defined over the same helical coordinate. Such an embodiment may be useful where a more detailed helical modulation structure is desired, such as when the local energy landscape is better represented by multiple periodic components rather than a single dominant harmonic. In narrower embodiments, an additional coupling coefficient such as beta_2 may be introduced to weight the higher-order term, and that coefficient may itself be bounded or derived from the same first-principles consistency framework used for the principal periodic coefficient beta. These higher-order harmonic embodiments preserve the same basic architecture of a paired-state potential modulated by helical periodicity, while allowing the periodic structure to be refined where appropriate.

In another class of alternative embodiments, the kinetic contribution may be generalized beyond the quadratic gradient term (kappa_DNA/2)*(dC/dr)\hat{ }2. For example, the kinetic term may be replaced or supplemented by a nonlinear term reflecting anharmonic stacking, torsional stress, stretch-induced nonlinearity, or other departures from the simplest gradient-cost model. In such embodiments, the local energetic penalty associated with spatial variation in C(r) may depend nonlinearly on dC/dr, on higher spatial derivatives, or on an additional deformation-related field coupled to C(r). This type of embodiment may be useful when the DNA duplex is modeled under mechanically stressed, stretched, or otherwise non-ideal conditions for which a strictly quadratic gradient penalty is insufficient. Even in such embodiments, however, the kinetic contribution remains directed to the same physical role described above, namely penalizing destabilizing spatial variation of the local base-pairing field along the modeled domain.

In further embodiments, the disclosed formulation may be extended to include explicit time dependence. In one such embodiment, a dynamic Lagrangian may be defined by introducing a time-derivative contribution such as L_dyn=(m_eff/2)*(partial C/partial t)\hat{ }2+L_DNA, where m_eff is an effective dynamic coefficient and L_DNA is the previously described spatial Lagrangian density. This embodiment may be useful for modeling transient or time-varying behavior, including relaxation, dynamic structural response, temporal perturbation propagation, or biological or design-environment evolution of the local pairing field. In such a time-dependent embodiment, the governing equation becomes a dynamical field equation rather than a purely static Euler-Lagrange boundary-value problem. Nevertheless, the same core architecture is preserved: the field C remains the principal state variable, the spatial stability structure remains derived from the disclosed DNA Lagrangian density, and the added time-dependent term serves only to extend the model into a dynamic regime.

In related embodiments, stochastic effects may be incorporated into the disclosed formulation. For example, noise, perturbation terms, stochastic forcing, Monte Carlo variation, or uncertainty-aware parameter sampling may be introduced in order to model environmental fluctuation, thermal perturbation, imperfect parameter knowledge, or probabilistic design screening. In such embodiments, the system may evaluate distributions of C(r), A, M_struct, M_melt, or related outputs rather than only a single deterministic profile. A stochastic embodiment may therefore be useful for robustness analysis, sensitivity analysis, or ranking candidate constructs under uncertain conditions. Such an embodiment remains within the disclosed invention because it continues to operate on the same field-theoretic structure and merely introduces uncertainty-aware evaluation of the same underlying Lagrangian-driven stability behavior.

The present disclosure also contemplates extensions beyond a strictly one-dimensional helical-coordinate treatment. In one alternative embodiment, the modeled field may be extended to a two-dimensional form or to a richer spatial representation that captures additional geometric degrees of freedom of the duplex. For example, the local state variable may depend on both the axial coordinate and a second coordinate associated with transverse, groove-related, or local-surface structure. A two-dimensional embodiment may be useful where a single helical coordinate is insufficient to capture the structural effect of local deformation, asymmetry, or coupled spatial modes. Even so, the resulting formulation would remain an equivalent implementation of the disclosed invention so long as it preserves the same conceptual structure of a field-based stability formulation with a rigidity contribution, a pairing preference, and a geometry-linked modulation.

The disclosed invention also preserves support for substitution of the DNA-specific parameter set with a corresponding parameter set for another nucleic-acid system. In one alternative embodiment, the formulation is applied to RNA stability by replacing DNA-specific parameters with RNA-specific parameters, including an RNA coherence-related energy scale, an RNA structural length scale, and, where relevant, an RNA reference pitch or corresponding RS-derived periodicity quantity. In another embodiment, the formulation is applied to peptide nucleic acid by substituting a corresponding PNA-specific parameter set. In each such case, the operative structure of the formulation remains the same: a local pairing-state field is defined over a modeled nucleic-acid domain, a Lagrangian density is formed from a rigidity contribution and a pairing-centered potential with periodic structure, and one or more stability outputs are computed therefrom. These substitution embodiments are useful because they preserve support for nucleic-acid analog implementations without changing the disclosed architecture of the invention.

Equivalent implementations are also contemplated with respect to computational realization. For example, the boundary-value problem may be solved analytically in a linearized regime, numerically by a boundary-value solver, iteratively by a finite-difference or finite-element scheme, or approximately by a perturbative or reduced-order method. Likewise, acceptance logic may be implemented through hard thresholds, weighted ranking, rule-based screening, learned decision layers constrained by the disclosed metrics, or combinations thereof. These variations do not alter the underlying invention because the core inventive content resides in the disclosed stability formulation and the derived quantities used to assess a candidate construct, not in any one exclusive numerical solver or software architecture.

These alternative embodiments are valuable from a robustness and no-CIP standpoint because they provide amendment room and equivalent-implementation support without departing from what is already directionally disclosed. A higher-order harmonic potential remains a refinement of the disclosed periodic modulation. A nonlinear kinetic term remains a refinement of the disclosed gradient-cost structure. A time-dependent extension remains an extension of the same field variable and the same spatial stability model. A stochastic or two-dimensional embodiment remains a richer implementation of the same stability-analysis architecture. RNA and PNA substitutions remain parameter substitutions into the same core formulation. Accordingly, these embodiments preserve prosecution flexibility while remaining within the conceptual and technical bounds of the presently disclosed invention.

Accordingly, the present disclosure contemplates alternative embodiments and equivalents including higher-order harmonic potential terms, nonlinear kinetic terms, time-dependent extensions, stochastic variants, richer spatial-domain formulations such as two-dimensional embodiments, and substitution of RNA-specific or PNA-specific parameter sets for the DNA-specific parameters described above. Each such embodiment preserves the essential structure of the disclosed field-based stability model and may be used where additional fidelity, alternative implementation, or nucleic-acid analog support is desired, while the central invention remains the first-principles Lagrangian-density formulation for predicting nucleic-acid structural stability through the behavior of a local pairing-state field.

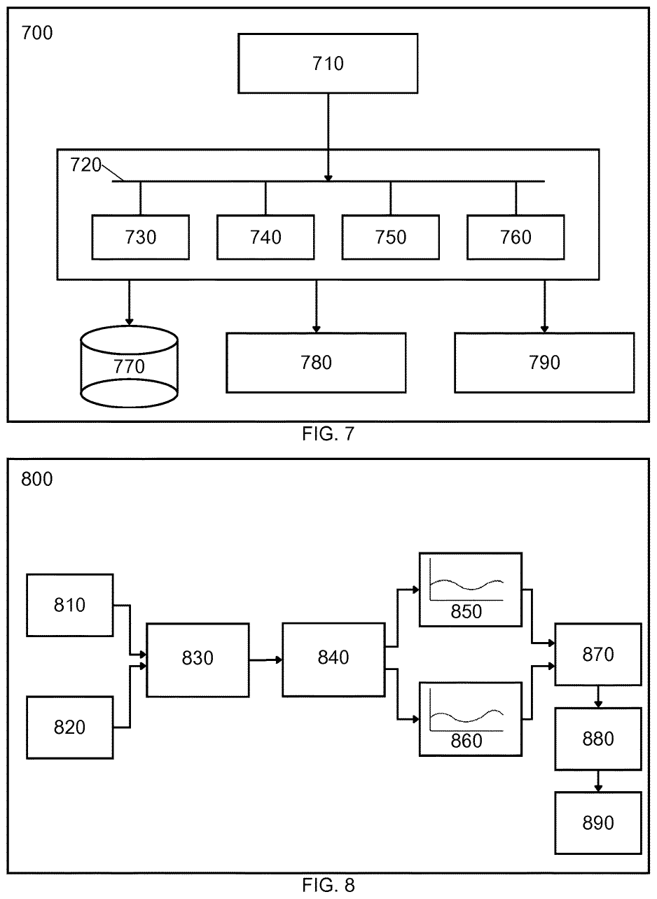

In some embodiments, the disclosed DNA structural stability formulation is implemented using a computing system (700) of the type shown in FIG. 7. The computing system (700) may include one or more processors (710), memory (720), parameter derivation instructions (730), solver instructions (740), metric computation instructions (750), decision logic instructions (760), a data store (770), a communication interface (780), and a user output interface (790). This computing-system support is provided to make clear that the present invention is not limited to a purely theoretical formulation, but may be embodied as executable computational logic that receives candidate DNA inputs, derives model coefficients, solves the disclosed field equation, computes stability metrics, and generates a technical output suitable for design validation, screening, ranking, or downstream integration.

The one or more processors (710) may execute instructions stored in memory (720) in order to perform the operations described throughout the present disclosure. The memory (720) may include any non-transitory machine-readable storage medium suitable for storing executable instructions, intermediate computational values, candidate sequence information, geometric input values, solver states, derived stability metrics, or result data. In some embodiments, the memory (720) stores the parameter derivation instructions (730), solver instructions (740), metric computation instructions (750), and decision logic instructions (760) as distinct software modules or callable routines. In other embodiments, those instructions may be integrated into a unified software package, a script-based computational workflow, a compiled program, a service-oriented architecture, or any technically suitable combination thereof.

The parameter derivation instructions (730) may cause the processor (710) to compute one or more model parameters used in the disclosed DNA Lagrangian density from incoming sequence-related and geometry-related data. For example, the parameter derivation instructions (730) may compute kappa_DNA=E_coh*X_DNA, a_bp=X_DNA/4, lambda_DNA=(E_bp/a_bp)*S_stab, and beta=E_coh*P0/(hbar*c) or another bounded periodic modulation coefficient. In embodiments using the optional groove-sensitive refinement, the parameter derivation instructions (730) may also determine gamma, G0, and any associated groove-deviation quantity needed for the optional correction term. Thus, the parameter derivation instructions (730) provide the executable mechanism by which raw or intermediate input values are transformed into the coefficient set used by the disclosed field-based stability formulation.

The solver instructions (740) may cause the processor (710) to construct and solve the governing equation associated with the DNA Lagrangian density. In disclosed embodiments, this may include forming the field equation over the interval r in [0,L], imposing boundary conditions such as C(0)=C(L)=1, computing the helical phase term cos(2*pi*r/P), and solving the resulting Euler-Lagrange equation analytically, approximately, or numerically. In one embodiment, the solver instructions (740) may invoke a boundary-value solver such as SciPy solve_bvp. In other embodiments, the solver instructions (740) may employ finite-difference methods, perturbative approximations, reduced-order methods, or other technically suitable numerical or symbolic solution approaches. The present invention is not limited to any one solver implementation, provided that the computing system (700) is able to determine a local base-pairing probability field C(r) or an equivalent solved stability representation from the disclosed formulation.

The metric computation instructions (750) may cause the processor (710) to derive one or more stability outputs from the solved field profile. In disclosed embodiments, these outputs may include the periodic amplitude A, the structural stability metric M_struct, the melting-barrier metric M_melt, and, in optional embodiments, a damage-resistance metric M_damage or a calibrated temperature-related output. The metric computation instructions (750) may also derive rank values, difference values for comparative-sequence embodiments, or application-specific summary values used in downstream reporting and selection. In this way, the metric computation instructions (750) convert the field solution into compact technical outputs usable by a design system, screening engine, or validation workflow.

The decision logic instructions (760) may cause the processor (710) to apply one or more of the disclosed operational envelopes and acceptance criteria. For example, the decision logic instructions (760) may determine whether |A|<=0.05, whether S_stab>=3, whether P in [30,40] Angstrom, whether G in [1.5,1.7], whether M_struct is below a selected threshold, and whether M_melt is above a selected threshold. In some embodiments, the decision logic instructions (760) may implement a binary pass/fail classifier. In other embodiments, the decision logic instructions (760) may implement weighted ranking, multi-stage filtering, transition-zone handling, or application-specific selection logic. As a result, the computing system (700) may not only solve the disclosed model, but may also transform the solved results into a concrete accept, reject, prioritize, or redesign determination.

The data store (770) may be used to retain candidate DNA sequences, candidate geometric inputs, derived parameter values, solved field profiles, computed metrics, acceptance histories, calibration values, or output reports. In some embodiments, the data store (770) may include a local memory region, a structured database, a file system, a cloud-based storage system, or another persistent or semi-persistent storage arrangement. The data store (770) may also support comparative analysis across multiple candidates by storing prior outputs, ranked candidate sets, or validation histories. This is particularly useful in embodiments involving DNARP integration, mutation comparison, or candidate ranking across a population of related constructs.

The communication interface (780) may permit the computing system (700) to exchange data with remote devices, design environments, DNARP pipelines, storage systems, or other computational services. Through the communication interface (780), the system may receive candidate input sets from an upstream design process, transmit solved stability outputs to a downstream screening or reporting system, or obtain externally generated geometric descriptors, calibration values, or comparative reference data. In some embodiments, the communication interface (780) supports a distributed implementation in which parameter derivation, solution, and reporting are performed on different machines or services. The present invention therefore contemplates both local and networked embodiments of the computing architecture.

The user output interface (790) may provide one or more technical outputs to a user, operator, application layer, or downstream automated process. Such outputs may include the local base-pairing probability field C(r), the amplitude A, the structural stability metric M_struct, the melting-barrier metric M_melt, an accept or reject decision, a rank-ordered candidate list, a mutation impact result, or an application-specific report. In some embodiments, the user output interface (790) may provide a stability score display, an acceptance indicator, a rejection indicator, or a generated validation report. In other embodiments, the interface may provide structured machine-readable outputs suitable for automated selection, filtering, or optimization in a larger computational design environment.

In some embodiments, system (100) of FIG. 1 may be implemented using the computing system (700) of FIG. 7. For example, the input interface (110) may correspond to one or more communication and input-handling components coupled to the computing system (700); the parameter derivation module (160) may be implemented by parameter derivation instructions (730); the Lagrangian construction module (170) and solver module (180) may be implemented by solver instructions (740); and the output module (190) may be implemented by metric computation instructions (750), decision logic instructions (760), and user output interface (790). In this way, FIG. 7 provides the computing architecture by which the functional system components of FIG. 1 may be realized in executable form.

The present disclosure is not limited to any specific processor type, programming language, operating system, memory arrangement, or deployment architecture. The computing system (700) may be implemented on a single workstation, a server, a cluster, a cloud-computing environment, a laboratory computing platform, or a distributed combination thereof. The solver, metric computation, and decision logic may be co-located or separated across networked resources. Likewise, the instructions may be embodied in software, firmware, hardware-assisted logic, or suitable combinations thereof. What is significant is that the disclosed computing system provides a concrete technical platform capable of carrying out the first-principles DNA stability analysis described herein.

Accordingly, FIG. 7 provides examiner-facing support for implementation of the disclosed DNA structural stability formulation as an executable computing system (700) including processors, memory, parameter derivation instructions, solver instructions, metric computation instructions, decision logic instructions, storage, communications, and output components. This computing-system support confirms that the present invention may be practiced as a concrete computational tool for receiving candidate DNA inputs, deriving the disclosed coefficients, solving the governing field equation, computing stability metrics, and generating a technical structural-stability result for use in validation, ranking, optimization, and downstream DNA design workflows.

The relationships used throughout the present disclosure are internally consistent with the disclosed first-principles parameter chain and the resulting field-based stability model. In particular, the stiffness parameter kappa_DNA remains positive under the disclosed relation kappa_DNA=E_coh*X_DNA, the sequence-scaled potential coefficient lambda_DNA remains positive for positive S_stab under the disclosed relation lambda_DNA=(E_bp/a_bp)*S_stab, and lambda_DNA increases monotonically with increasing sequence stability metric S_stab. Likewise, the periodic modulation coefficient beta is treated as bounded in the interval 0<beta<1, so that the helical periodic contribution remains a modulation factor rather than a dominant instability source. These consistency properties support the disclosed interpretation that the DNA Lagrangian density is constructed from positive rigidity, positive pairing preference, and bounded helical forcing rather than from unconstrained fitting terms.

The same consistency carries through to the disclosed stability metrics. The structural stability metric M_struct=integral_0\hat{ }L (1−C(r))\hat{ }2 dr is non-negative because its integrand is non-negative over the modeled domain, and M_struct vanishes only when the local base-pairing probability field remains at the fully paired condition throughout the interval. The melting-barrier metric M_melt=lambda_DNA(S_stab)*L/2 is positive for positive lambda_DNA and positive L, and increases monotonically with increasing S_stab through the monotone dependence of lambda_DNA on sequence stability. These properties reinforce that the disclosed outputs are not arbitrary reporting values, but analytically consistent quantities derived from the same field-theoretic structure used to generate the stability profile.

The disclosed formulation is also consistent with the present invention's non-empirical character. The principal coefficients kappa_DNA and lambda_DNA are derived from the disclosed RS-derived quantities E_coh, E_bp, and X_DNA, while beta is treated as a bounded coupling with a disclosed RS base estimate. As a result, the core DNA Lagrangian density is not dependent on experimentally fitted DNA melting tables in order to define its operative coefficient structure. This consistency is useful because it confirms that the formulation, the resulting field equation, and the derived metrics all remain aligned with the disclosed first-principles derivation chain and with the claimed role of the invention as a structural-stability engine for DNA analysis, validation, and design workflows.

What is claimed is:

1. A computer-implemented method for predicting structural stability of a deoxyribonucleic acid (DNA) construct, the method comprising: receiving or determining a sequence stability metric S_stab, a sequence extent L, a helical pitch P, a groove ratio G, and Recognition Science-derived parameters including E_coh, E_bp, and X_DNA; computing a stiffness parameter kappa_DNA from E_coh and X_DNA, an effective base-pair axial scale a_bp from X_DNA, a sequence-scaled potential coefficient lambda_DNA from E_bp, a_bp, and S_stab, and a periodic modulation coefficient beta; constructing a DNA Lagrangian density that acts on a local base-pairing probability field C(r) over a helical coordinate interval r in [0,L], the DNA Lagrangian density including a kinetic term weighted by kappa_DNA and a potential term including a pairing contribution centered at C=1 and a periodic modulation contribution varying with P;
solving an Euler-Lagrange equation associated with the DNA Lagrangian density subject to boundary conditions C(0)=C(L)=1 to generate a structural stability profile C(r) for the DNA construct; computing at least one stability metric from the structural stability profile C(r); and
outputting a structural stability result for technical evaluation of the DNA construct.

2. The method of claim 1, wherein computing the stiffness parameter comprises computing kappa_DNA=E_coh*X_DNA.

3. The method of claim 2, wherein computing the effective base-pair axial scale and the sequence-scaled potential coefficient comprises computing a_bp=X_DNA/4 and lambda_DNA=(E_bp/a_bp)*S_stab.

4. The method of claim 3, wherein computing the periodic modulation coefficient comprises computing beta from E_coh, a reference pitch P0, hbar, and c, and wherein beta is bounded such that 0<beta<1.

5. The method of claim 1, wherein the potential term further includes a groove-sensitive correction term scaled by a groove correction coefficient gamma and dependent on a departure of G from a reference groove ratio G0.

6. The method of claim 1, wherein generating the structural stability profile C(r) comprises determining a periodic amplitude A according to A=beta*lambda_DNA/(kappa_DNA*(2*pi/P)\hat{ }2+lambda_DNA).

7. The method of claim 1, wherein computing the at least one stability metric comprises computing a structural stability metric M_struct=integral_0\hat{ }L (1<C(r))\hat{ }2 dr and a melting-barrier metric M_melt=lambda_DNA*L/2.

8. The method of claim 7, wherein outputting the structural stability result comprises classifying the DNA construct as acceptable only when abs(A)<=delta_A, wherein A is a periodic amplitude of the structural stability profile C(r), S_stab>=3, P is within 30 Angstrom to 40 Angstrom, G is within 1.5 to 1.7, M_struct satisfies a predetermined structural criterion, and M_melt satisfies a predetermined melting-barrier criterion.

9. A DNA structural stability prediction system, comprising: one or more processors; memory storing instructions; and an output interface, wherein execution of the instructions by the one or more processors causes the system to receive or determine a sequence stability metric S_stab, a sequence extent L, a helical pitch P, a groove ratio G, and Recognition Science-derived parameters including E_coh, E_bp, and X_DNA, compute kappa_DNA, a_bp, lambda_DNA, and beta, construct a DNA Lagrangian density acting on a local base-pairing probability field C(r) over r in [0,L], solve an Euler-Lagrange equation associated with the DNA Lagrangian density subject to boundary conditions C(0)=C(L)=1 to generate a structural stability profile C(r), compute at least one stability metric from the structural stability profile C(r), and output a structural stability result for the DNA construct.

10. The system of claim 9, wherein the instructions include a parameter derivation module, a solver module, a metrics module, a decision module, and an output module.

11. The system of claim 10, further comprising a data store configured to store candidate DNA sequences, geometric input values, solved field profiles, computed metrics, or validation histories, and a communication interface configured to exchange data with a remote design environment.

12. The system of claim 11, wherein the structural stability result comprises at least one of a predicted field profile, an amplitude value, a structural stability metric, a melting-barrier metric, an acceptance indication, a rejection indication, a rank-ordered candidate output, or a generated report.

13. The system of claim 9, wherein the system is configured as a DNA stability evaluation engine within a DNARP pipeline to evaluate a candidate object including a sequence component S and a shape component H.

14. The system of claim 13, wherein the system is further configured to receive a reference sequence and a variant sequence, generate a reference stability profile and a variant stability profile, compute a differential stability metric between the reference stability profile and the variant stability profile, and output a mutation impact result.

15. A non-transitory computer-readable medium storing instructions that, when executed by one or more processors, cause the one or more processors to: receive or determine a sequence stability metric S_stab, a sequence extent L, a helical pitch P, a groove ratio G, and Recognition Science-derived parameters including E_coh, E_bp, and X_DNA; compute kappa_DNA, a_bp, lambda_DNA, and beta; construct a DNA Lagrangian density acting on a local base-pairing probability field C(r) over r in [0,L]; solve an Euler-Lagrange equation associated with the DNA Lagrangian density subject to boundary conditions C(0)=C(L)=1 to generate a structural stability profile C(r); compute at least one stability metric from the structural stability profile C(r); and output a structural stability result for a DNA construct.

16. The non-transitory computer-readable medium of claim 15, wherein the instructions cause the one or more processors to solve the Euler-Lagrange equation as a boundary-value problem.

17. The non-transitory computer-readable medium of claim 16, wherein solving the Euler-Lagrange equation comprises generating the structural stability profile in a linearized form:
$$C ( r ) = 1 - A cos ( 2 pi * r / P ) + epsilon\_decay ( r ) ,$$
wherein A is a periodic amplitude and epsilon_decay(r) is an end-effect correction associated with a finite interval and imposed boundary conditions.

18. The non-transitory computer-readable medium of claim 17, wherein epsilon_decay(r) decays with distance from at least one boundary of the interval r in [0, L], such that the structural stability profile satisfies the boundary conditions while remaining approximately periodic within an interior portion of the interval.

19. The method of claim 1, wherein receiving or determining the sequence stability metric S_stab comprises computing S_stab=w_CGf_CG+w_ATf_AT, where f_CG and f_AT represent respective CG and AT fractions of the DNA construct over a selected sequence interval, and w_CG and w_AT are selected weighting constants.

20. The non-transitory computer-readable medium of claim 16, wherein solving the Euler-Lagrange equation as the boundary-value problem comprises invoking a numerical boundary-value solver, a finite-difference solver, or a finite-element solver.
